SEQUENCE LISTING (37 sequences)

SEQ ID NO: 1 - protein, 58 aa, synthetic construct
MQMFPPSPLFFFLQLLKQSSRRLEHTFVFLRNFSLMLLRGIGKKRRATRFWDPRRGTP

SEQ ID NO: 2 - protein, 9 aa, synthetic construct
FVFLRNFSL

SEQ ID NO: 3 - protein, 9 aa, synthetic construct
FLRNFSLMV

SEQ ID NO: 4 - protein, 20 aa, synthetic construct
MQMFPPSPLFFFLQLLKQSS

SEQ ID NO: 5 - protein, 20 aa, synthetic construct
FFLQLLKQSSRRLEHTFVFL

SEQ ID NO: 6 - protein, 20 aa, synthetic construct
RRLEHTFVFLRNFSLMLLRG

SEQ ID NO: 7 - protein, 20 aa, synthetic construct
RNFSLMLLRGIGKKRRATRF

SEQ ID NO: 8 - protein, 18 aa, synthetic construct
IGKKRRATRFWDPRRGTP

SEQ ID NO: 9 - protein, 9 aa, synthetic construct
  SITE 1: note = Palmitoyl modified residue
FVFLRNFSL

SEQ ID NO: 10 - protein, 9 aa, synthetic construct
  SITE 1: note = Palmitoyl modified residue
FLRNFSLMV

SEQ ID NO: 11 - protein, 10 aa, synthetic construct
  SITE 1: note = Palmitoyl modified residue
FPPSPLFFFL

SEQ ID NO: 12 - protein, 20 aa, synthetic construct
  SITE 1: note = Palmitoyl modified residue
MQMFPPSPLFFFLQLLKQSS

SEQ ID NO: 13 - protein, 20 aa, synthetic construct
  SITE 1: note = Palmitoyl modified residue
RRLEHTFVFLRNFSLMLLRG

SEQ ID NO: 14 - protein, 20 aa, synthetic construct
  SITE 1: note = Palmitoyl modified residue
RNFSLMLLRGIGKKRRATRF

SEQ ID NO: 15 - protein, 18 aa, synthetic construct
  SITE 1: note = Palmitoyl modified residue
IGKKRRATRFWDPRRGTP

SEQ ID NO: 16 - protein, 20 aa, synthetic construct
  SITE 1: note = Palmitoyl modified residue
LQLLKQSSRRLEHTFVFLRR

SEQ ID NO: 17 - protein, 16 aa, synthetic construct
  REGION 1..7: note = Anionic amino acids
SSEEEDEFVFLRNFSL

SEQ ID NO: 18 - protein, 16 aa, synthetic construct
  REGION 1..7: note = Anionic amino acids
SSEEEDEFLRNFSLMV

SEQ ID NO: 19 - protein, 27 aa, synthetic construct
  REGION 1..7: note = Anionic amino acids
SSEEEDEMQMFPPSPLFFFLQLLKQSS

SEQ ID NO: 20 - protein, 27 aa, synthetic construct
  REGION 1..7: note = Anionic amino acids
SSEEEDEFFLQLLKQSSRRLEHTFVFL

SEQ ID NO: 21 - protein, 27 aa, synthetic construct
  REGION 1..7: note = Anionic amino acids
SSEEEDERRLEHTFVFLRNFSLMLLRG

SEQ ID NO: 22 - protein, 27 aa, synthetic construct
  REGION 1..7: note = Anionic amino acids
SSEEEDERRLEHTFVFLRNFSLMVLRG

SEQ ID NO: 23 - protein, 27 aa, synthetic construct
  REGION 1..7: note = Anionic amino acids
SSEEEDERNFSLMLLRGIGKKRRATRF

SEQ ID NO: 24 - protein, 25 aa, synthetic construct
  REGION 1..7: note = Anionic amino acids
SSEEEDEIGKKRRATRFWDPRRGTP

SEQ ID NO: 25 - protein, 19 aa, synthetic construct
  REGION 1..8: note = Anionic amino acids
SSEEEDEEHTFVFLRNFSL

SEQ ID NO: 26 - protein, 18 aa, synthetic construct
  REGION 1..7: note = Anionic amino acids
SSEEEDEFVFLRNFSLMV

SEQ ID NO: 27 - protein, 38 aa, synthetic construct
MQMFPPSPLFFFLQLLKQSSRRLEHTFVFLRNFSLMVL

SEQ ID NO: 28 - protein, 32 aa, synthetic construct
FVFLRNFSLMVLRGIGKKRRATRFWDPRRGTP

SEQ ID NO: 29 - protein, 45 aa, synthetic construct
  REGION 1..8: note = Anionic amino acids
SEEEDESSMQMFPPSPLFFFLQLLKQSSRRLEHTFVFLRNFSLMV

SEQ ID NO: 30 - protein, 42 aa, synthetic construct
  REGION 1..10: note = Anionic amino acids
SEEEDESEEDFVFLRNFSLMVLRGIGKKRRATRFWDPRRGTP

SEQ ID NO: 31 - protein, 10 aa, synthetic construct
FLPSPLFFFL

SEQ ID NO: 32 - protein, 46 aa, synthetic construct
  REGION 1..8: note = Anionic amino acids
SEEEDESSMQMFPPSPLFFFLQLLKQSSRRLEHTFVFLRNFSLMLV

SEQ ID NO: 33 - protein, 7 aa, synthetic construct
SSEEEDE

SEQ ID NO: 34 - protein, 8 aa, synthetic construct
SSEEEDEE

SEQ ID NO: 35 - protein, 8 aa, synthetic construct
SEEEDESS

SEQ ID NO: 36 - protein, 10 aa, synthetic construct
SEEEDESEED

SEQ ID NO: 37 - protein, 19 aa, synthetic construct
  SITE 1: note = Palmitoyl modified residue
FFLQLLKQSSRRLEHTFVF

(19) United States
(12) Patent Application Publication
(10) Pub. No.: US 2024/0317833 A1
(43) Pub. Date: Sep. 26, 2024

(54) NON-HLA RESTRICTED T CELL VACCINE FOR TCR GAMMA ALTERNATE READING FRAME PROTEIN (TARP) PROTEIN EXPRESSING CANCERS

(71) Applicant: PDS BIOTECHNOLOGY CORPORATION, Princeton, NJ (US)

(72) Inventors: Frank Bedu-Addo, Stamford, CT (US); Joseph Dervan, Doylestown, PA (US); Gregory Conn, Madrid (ES)

(21) Appl. No.: 18/381,081

(22) Filed: Oct. 17, 2023

Related U.S. Application Data
(60) Provisional application No. 63/416,899, filed on Oct. 17, 2022.

(51) Int. Cl. C07K 14/725; A61K 39/00; A61K 45/06; A61P 35/00

(52) U.S. Cl. CPC C07K 14/7051; A61K 45/06; A61P 35/00; A61K 2039/55555; A61K 2039/55572; A61K 2039/70

(57) ABSTRACT

Novel compositions of TCR Gamma Alternate Reading Frame Protein (TARP) peptides combined with cationic lipids such as the DOTAP and specifically R-DOTAP, induce high levels of TARP-specific polyfunctional cytolytic T-cells. Compositions and methods of use are provided. The compositions comprise N-terminal and C-terminal overlapping peptide sequence pairs duplicating the critical central antigenic region of TARP and encompassing the entire protein selected and designed to be effectively processed by antigen-presenting cells to prime cytotoxic T cells specific for TARP-derived T cell peptide antigens when delivered in combination with immunostimulatory nanoparticles composed of R-DOTAP cationic lipids.

### CROSS-REFERENCE TO RELATED APPLICATIONS

This application claims benefit of priority under 35 U.S.C. § 119(e) of U.S. Provisional Application No. 63/416,899, filed Oct. 17, 2022. The disclosure of the prior applications is considered part of and are herein incorporated by reference in the disclosure of this application in their entirety.

### INCORPORATION OF SEQUENCE LISTING

The material in the accompanying sequence listing is hereby incorporated by reference into this application. The accompanying sequence listing xml file, name ST26.xml, was created on and is kb.

### BACKGROUND OF THE INVENTION

### Field of the Invention

The present invention relates generally to anti-cancer vaccines, and more specifically to non-HLA restricted multiepitope TARP vaccine compositions including cationic lipids.

### Background Information

Historically five categories of tumor antigens have been utilized in immunotherapy: mutated antigens (e.g., p53 or RAS), overexpressed self-antigens (e.g., HER2/neu), differentiation antigens (e.g., gplOO, tyrosinase), cancer testis antigens (e.g., MAGE, BAGE or CAGE families, NYESO1) and viral antigens (e.g., HPV16 E6 or E7, EBV). The advantages of therapeutic cancer vaccines utilizing proteins and peptides include the simplicity of production and the relative absence of major safety and regulatory issues. All cells that express major histocompatibility complex (MHC) class I molecules can present short peptides from tumor associated antigens or viruses whose chronic infection is associated with malignancy (e.g., human papilloma virus, hepatitis B virus, and hepatitis C virus). However, signals essential for T cell stimulation and the induction of lasting potent and effective immune responses are often absent, resulting in suboptimal CD8⁺ T-cell responses caused by a lack of proper cell mediated signaling through dendritic cells (DCs). Utilization of R-DOTAP as an immune-potentiating agent in vaccines has been proven to resolve this problem, producing strong, effective immune responses with these types of peptide antigens.

The TARP (T-cell receptor alternate reading frame) protein is a 58 amino acid protein identified using an expressed sequence database. The mRNA for the protein is initiated in the Jy 1 exon of the T-cell receptor (TCR) γ sequence and the protein expressed is initiated in an alternative reading frame distinct from that of the TCR γ coding sequence. TARP is highly expressed in primary as well as metastatic prostate cancer and other cancers; and is expressed in both hormone sensitive and castrate resistant prostate cancer. TARP is expressed by both normal and malignant prostate tissue and is overexpressed in 95% of prostate cancer specimens prostate and ˜50% of breast cancers, with very little or no expression in normal cells, making it a good target antigen for therapeutic vaccination. TARP is over-expressed in ˜90% of prostate and ˜50% of breast cancers, with very little or no expression in normal cells. Engineered CD8⁺ T cells recognizing the TARP derived T cell epitope can kill, in an antigen-dependent manner, tumor cells expressing TARP proteins.

Combinations of immunotherapeutic treatments, for example immune checkpoint inhibitors combined with therapeutic cancer vaccines, are rapidly gaining interest as approaches to treat cancer. A critical component of combination cancer immunotherapeutic treatments is activation, amplification and targeting of cancer antigen-specific cytotoxic T-cells; these treatments play their crucial role in modulating the host immune system to recognize tumor-associated antigens and trigger cellular antitumor immune responses to eliminate targeted cancer cells. There are several approaches being developed for novel T-cell-activating cancer-targeting immunotherapeutics; 1) whole tumor cell-based immunotherapeutics, combining tumor cell components with immunostimulatory adjuvant compounds, 2) similar recombinant tumor antigen protein/peptide-based immunotherapeutics, and 3) immunotherapeutics based on DNA/RNA coding target tumor antigens. In all other previous examples developed to date the various immunostimulatory adjuvants utilized in these therapeutics, including oil in water emulsions, alum particles, and various polysaccharide and lipid components, have failed to induce strong and effective, yet safe, polyfunctional cytotoxic T cell responses in patients. There are several important hurdles that such a therapeutic cancer immunotherapeutic must overcome to induce antitumor immunity. The first hurdle is the correct choice of tumor antigen. Ideal candidates for this are antigens that are exclusively expressed in tumors, such as tumor-specific antigens (TSA), or antigens that are uniquely altered in the tumor cells, such as neoantigens or tumor-associated antigens (TAA). The second hurdle to overcome is the ability to effectively present these antigens to the dendritic cells to enable more effective presentation both via MHC I (CD8⁺ T cells) and MHC II (CD4 T cells). Currently utilized approaches and immune adjuvants have been ineffective in this regard. The third hurdle for an effective immunotherapeutic is to activate T cell immune responses in a broad targeted patient population expressing a wide range of HLA subtypes.

Cationic lipids are unique in their ability to rapidly bind to antigen-presenting dendritic cells in a receptor-independent fashion and be taken up into early endosomes along with associated peptide or protein antigens. These specific cationic lipids are capable of facilitating orders of magnitude more protein and peptide antigens entry into the MHC class I and MHC class II pathways than other current approaches such as the use of alum or oil in water adjuvants to induce dendritic cell maturation. Furthermore, vaccination with these peptide-loaded cationic lipid nanoparticles induces superior T cell immune responses in vivo compared to peptide alone or peptides formulated with traditional adjuvants.

The current invention provides a highly effective and safe cancer immunotherapy to treat human cancers expressing TARP tumor associated antigen in an HLA unrestricted manner.

### SUMMARY OF THE INVENTION

The present invention is based on the seminal discovery of multiepitope TARP peptides with overlapping amino acid sequences used in vaccine compositions with cationic lipid as adjuvants induce non-HLA-restricted multifunction anti-cancer specific T cells responses.

In one embodiment, the present invention provides a multiepitope peptide including at least one multiepitope peptide having an amino acid sequence with at least 80%, 85%, 90% or 95% sequence identity with the amino acid sequence of any one of SEQ ID NOs:27-30 and 32.

In one aspect, the at least one multiepitope peptide is modified. In one aspect, the at least one multiepitope peptide is covalently modified. In one aspect, the modification includes palmitoylation or addition of an anionic sequence.

In one aspect, the at least one multiepitope peptide is modified. In one aspect, the at least one multiepitope peptide is covalently modified. In one aspect, the modification includes palmitoylation or addition of an anionic sequence.

In one embodiment, the present invention provides a composition including one or more multiepitope peptides having an amino acid sequence with at least 80%, 85%, 90% or 95% sequence identity with the amino acid sequence of any one of SEQ ID NOs:27-30 and 32 and an adjuvant, wherein the adjuvant is a cationic lipid.

In one aspect, the cationic lipid is DOTAP, DDA, DOEPC, DOTMA, R-DOTAP, R-DDA, R-DOEPC, R-DOTMA, S-DOTAP, S-DDA, S-DOEPC, S-DOTMA, variations thereof or analogs thereof. In one aspect, the cationic lipid is R-DOTAP.

In one aspect, the one or more multiepitope peptides are modified by palmitoylation or by the addition of at least one anionic amino acid. In one aspect, the one or more multiepitope peptides are encapsulated within cationic liposomes or mixed as separate micelles with preformed cationic lipid nanoparticles. In one aspect, the one or more multiepitope peptides and the preformed cationic lipid nanoparticles are mixed at a 1:1 ratio.

In one aspect, the composition includes an enhancer agonist epitope and/or an analog thereof.

In one embodiment, the present invention provides a vaccine composition including a multiepitope peptide and an adjuvant, wherein the adjuvant is a cationic lipid, wherein the multiepitope peptide includes at least one TARP peptide.

In one aspect, the multiepitope peptide have at least one multiepitope peptide including an amino acid sequence with at least 80%, 85%, 90% or 95% sequence identity with the amino acid sequence of any one of SEQ ID NOs:27-30 and 32.

In one aspect, the at least one multiepitope peptide has an amino acid sequence with at least 80%, 85%, 90% or 95% sequence identity with any one of SEQ ID NOs:27 and 28.

In one aspect, the at least one multiepitope peptide has an amino acid sequence with at least 80%, 85%, 90% or 95% sequence identity with SEQ ID NO:27 and SEQ ID NO:28. In one aspect, the at least one multiepitope peptide has an amino acid sequence of SEQ ID NOs:27 and 28. In one aspect, the at least one multiepitope peptide has an amino acid sequence of SEQ ID NO:27. In one aspect, the at least one multiepitope peptide has an amino acid sequence of SEQ ID NO: 28. In one aspect, the at least one TARP peptide includes an amino acid sequence comprising any of SEQ ID NOs:2-8.

In one aspect, the at least one multiepitope peptide is modified by palmitoylation or by the addition of an anionic amino acid sequence.

In one aspect, the at least one multiepitope peptide cover a T-cell receptor alternate reading frame protein (TARP) amino acid sequence. In one aspect, the at least one multiepitope peptide binds to CD4+ T cells and/or CD8+ T cells.

In one aspect, the at least one multiepitope peptide is modified. In one aspect, the at least one multiepitope peptide is covalently modified. In one aspect, modification includes palmitoylation or addition of an anionic sequence.

In one aspect, the cationic lipid is DOTAP, DDA, DOEPC, DOTMA, R-DOTAP, R-DDA, R-DOEPC, R-DOTMA, S-DOTAP, S-DDA, S-DOEPC, S-DOTMA, variations thereof or analogs thereof. In one aspect, the cationic lipid is R-DOTAP.

In one aspect, the at least one multiepitope peptide is encapsulated in a cationic liposome or mixed as micelles separately with preformed cationic lipid nanoparticles. In one aspect, the at least one multiepitope peptide and the preformed cationic lipid nanoparticles are mixed at a 1:1 ratio.

In one embodiment, the present invention provides a method of treating cancer in a subject including administering to the subject the vaccine composition including at least one multiepitope peptide, wherein the multiepitope peptide includes at least one TARP peptide; and an adjuvant, wherein the adjuvant is a cationic lipid.

Having at least one multiepitope peptide including an amino acid sequence with at least 80%, 85%, 90% or 95% sequence identity with the amino acid sequence of any one of SEQ ID NOs:27-30 and 32

In one aspect, the at least one multiepitope peptide has an amino acid sequence with at least 80%, 85%, 90% or 95% sequence identity with any one of SEQ ID NOs:27-30 and 32. In one aspect, the at least one multiepitope peptide has an amino acid sequence with at least 80%, 85%, 90% or 95% sequence identity with any one of SEQ ID NOs:27 and 28. In one aspect, the at least one multiepitope peptide has an amino acid sequence with at least 80%, 85%, 90% or 95% sequence identity with SEQ ID NO:27 and SEQ ID NO:28. In one aspect, the at least one multiepitope peptide has an amino acid sequence of SEQ ID NOs:27 and 28. In one aspect, the at least one multiepitope peptide has an amino acid sequence of SEQ ID NO:27. In one aspect, the at least one multiepitope peptide has an amino acid sequence of SEQ ID NO: 28. In one aspect, the at least one TARP peptide includes an amino acid sequence comprising any of SEQ ID NOs:2-8.

In one aspect, the at least one multiepitope peptide cover a T-cell receptor alternate reading frame protein (TARP) amino acid sequence. In one aspect, the at least one multiepitope peptide is modified. In one aspect, the modification includes palmitoylation or addition of an anionic sequence.

In one aspect, the cationic lipid is DOTAP, DDA, DOEPC, DOTMA, R-DOTAP, R-DDA, R-DOEPC, R-DOTMA, S-DOTAP, S-DDA, S-DOEPC, S-DOTMA, variations thereof or analogs thereof. In one aspect, the cationic lipid is R-DOTAP.

In one aspect, the at least one multiepitope peptide is encapsulated with cationic lipid nanoparticles or mixed as separate micelles with pre-formed cationic lipid nanoparticles. In one aspect, the at least one multiepitope peptide and the preformed cationic lipid nanoparticles are mixed at a 1:1 ratio.

In one aspect, the at least one multiepitope peptide induces the presentation of non-HLA restricted peptides to CD4⁺ and CD8⁺ T cells by antigen presenting cells. In one aspect, treating cancer includes preventing progression of cancer in the subject. In one aspect, the cancer includes TARP expressing cancer cells. In one aspect, the cancer is prostate cancer, breast cancer or acute myeloid leukemia (AML). In one aspect, the method includes administering to the subject an anti-cancer treatment. In some aspects, the anti-cancer treatment includes surgery, radiotherapy, chemotherapy, immunotherapy, targeted therapy, or any combination thereof. In various aspects, the immunotherapy includes an immune checkpoint inhibitor therapy. In many aspects, the checkpoint inhibitor therapy includes a programmed cell death 1 protein (PD-1) inhibitor, a PD-1 ligand 1 (PD-L1) inhibitor, and/or a cytotoxic T-lymphocyte-associated protein 4 (CTLA-4) inhibitor. In other aspects, the targeted therapy comprises a histone deacetylase (HDAC) inhibitor. In one aspect, treating cancer includes inducing a TARP-specific polyfunctional cytolytic T cell response in the subject.

In one embodiment, the present invention provides a method of inducing a TARP-specific polyfunctional cytolytic T cell response in the subject including administering to the subject the vaccine composition including at least one multiepitope peptides including at least one multiepitope peptide including at least one TARP peptide; and an adjuvant, wherein the adjuvant is a cationic lipid. In one aspect, the at least one multiepitope peptide includes an amino acid sequence with at least 80%, 85%, 90% or 95% sequence identity with any one of SEQ ID NOs: 27-30 and 32. In one aspect, the at least one multiepitope peptide includes an amino acid sequence with at least 80%, 85%, 90% or 95% sequence identity with SEQ ID NO:27 and SEQ ID NO:28. In one aspect, the at least one multiepitope peptide includes an amino acid sequence of SEQ ID NOs:27 and 28. In one aspect, the at least one multiepitope peptide includes an amino acid sequence of SEQ ID NO:27. In one aspect, the at least one multiepitope peptide includes an amino acid sequence of SEQ ID NO: 28. In one aspect, the at least one TARP peptide includes an amino acid sequence comprising any of SEQ ID NOs:2-8. In one aspect, the at least one multiepitope peptide covers a T-cell receptor alternate reading frame protein (TARP) amino acid sequence. In one aspect, the at least one multiepitope peptide is modified. In one aspect, the modification comprises palmitoylation or addition of an anionic sequence. In one aspect, the cationic lipid is DOTAP, DDA, DOEPC, DOTMA, R-DOTAP, R-DDA, R-DOEPC, R-DOTMA, S-DOTAP, S-DDA, S-DOEPC, S-DOTMA, variations thereof or analogs thereof. In one aspect, the cationic lipid is R-DOTAP. In one aspect, the at least one multiepitope peptide is encapsulated with cationic lipid nanoparticles or mixed as separate micelles with preformed cationic lipid nanoparticles. In one aspect, wherein the at least one multiepitope peptide and the preformed cationic lipid nanoparticles are mixed at a 1:1 ratio. In one aspect, the at least one multiepitope peptide induces the presentation of non-HLA restricted peptides to CD4⁺ and CD8⁺ T cells by antigen presenting cells.

### BRIEF DESCRIPTION OF THE DRAWINGS

FIG. 1 is a graph illustrating the efficacy of vaccine formulations in generating T cell responses as measured by dosing the number of IFNγ producing cells. TARP. Suc mice were vaccinated with TARP peptides (SEQ ID NO:27 and SEQ ID NO:28) formulated in sucrose solution. Ace: The two long overlapping peptides synthesized with acetate counter ion. HCL: The two long overlapping peptides synthesized with HCL counter ion.

FIG. 2 is a graph illustrating the efficacy of vaccine formulations in generating T cell responses as measured by dosing the number of IFNγ producing cells.

FIG. 3 is a graph illustrating the efficacy of vaccine formulations in generating T cell responses as measured by dosing the number of IFNγ producing cells. Anion. TARP: a mixture of SEQ ID NO:29 and SEQ ID NO:30) TARP-Suc: Sucrose control mice were vaccinated with TARP peptides (SEQ ID NO:11 and SEQ ID NO:12) formulated in sucrose solution alone. TARP. CFA: mice were vaccinated with TARP peptides (SEQ ID NO:27 and SEQ ID NO:28) formulated in Freund's adjuvant.

FIGS. 4A-4C illustrate T cell response induction by long TARP peptides mixtures. FIG. 4A is a graph illustrating the efficacy of vaccine formulations in generating T cell responses as measured by dosing the number of IFNγ producing cells. FIG. 4B is a graph illustrating the efficacy of vaccine formulations in generating T cell responses as measured by dosing the number of IFNγ producing cells. FIG. 4C is a graph illustrating the efficacy of vaccine formulations in generating T cell responses as measured by dosing the number of IFNγ producing cells.

FIG. 5 is a graph illustrating the efficacy of vaccine formulations in generating T cell responses as measured by dosing the number of IFNγ producing cells.

FIG. 6 is a graph illustrating the efficacy of vaccine formulations in generating T cell responses as measured by dosing the number of IFNγ producing cells.

FIG. 7 is a graph illustrating the efficacy of vaccine formulations in generating T cell responses as measured by dosing the number of IFNγ producing cells.

FIG. 8 is a graph illustrating the efficacy of vaccine formulations in generating T cell responses as measured by dosing the number of IFNγ producing cells.

FIG. 9 is a graph illustrating the efficacy of vaccine formulations in generating T cell responses as measured by dosing the number of IFNγ producing cells.

FIG. 10 is a graph illustrating the efficacy of vaccine formulations in generating T cell responses as measured by dosing the number of IFNγ producing cells. x-axis: peptide vaccine mixtures.

FIG. 11 is a graph illustrating the efficacy of vaccine formulations in generating T cell responses as measured by dosing the number of IFNγ producing cells. x-axis: peptide vaccine mixtures.

FIG. 12 is a graph illustrating the efficacy of vaccine formulations in generating T cell responses as measured by dosing the number of IFNγ producing cells. x-axis: peptide vaccine mixtures.

FIG. 13 is a graph illustrating the efficacy of vaccine formulations in generating T cell responses as measured by dosing the number of IFNγ producing cells. x-axis: peptide vaccine mixtures.

FIG. 14 is a graph illustrating the efficacy of vaccine formulations in generating T cell responses as measured by dosing the number of IFNγ producing cells. x-axis: peptide vaccine mixtures.

FIG. 15 is a graph illustrating the efficacy of vaccine formulations in generating T cell responses as measured by dosing the number of IFNγ producing cells. x-axis: peptide vaccine mixtures.

FIG. 16 is a graph illustrating the efficacy of vaccine formulations in generating T cell responses as measured by dosing the number of IFNγ producing cells. x-axis: peptide vaccine mixtures.

FIG. 17 is a graph illustrating the efficacy of vaccine formulations in generating T cell responses as measured by dosing the number of IFNγ producing cells. x-axis: peptide vaccine mixtures.

### DETAILED DESCRIPTION

The present invention is based on the seminal discovery of multiepitope TARP peptides with overlapping amino acid sequences used in vaccine compositions with cationic lipid as adjuvants induce non-HLA-restricted multifunction anti-cancer specific T cells responses.

Before the present compositions and methods are described, it is to be understood that this invention is not limited to particular compositions, methods, and experimental conditions described, as such compositions, methods, and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

As used herein, the term “and/or” includes any and all combinations of one or more of the associated listed items.

As used herein, the term “about” in association with a numerical value is meant to include any additional numerical value reasonably close to the numerical value indicated. For example, and based on the context, the value can vary up or down by 5-10%. For example, for a value of about 100, means 90 to 110 (or any value between 90 and 110).

All publications, patents, and patent applications mentioned in this specification are herein incorporated by reference to the same extent as if each individual publication, patent, or patent application was specifically and individually indicated to be incorporated by reference.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, it will be understood that modifications and variations are encompassed within the spirit and scope of the instant disclosure. The preferred methods and materials are now described.

### Overview

Disclosed herein are methods for the design and use of unique long peptide sequences, including (SEQ ID NO:27) and (SEQ ID NO:28), derived from TARP protein. These two long novel peptides consist of an N-terminal and a C-terminal TARP peptide of 32-38 amino acids that share a 12 aa overlap, at the C-terminus and N-terminus of each peptide respectively, which includes an important immunogenic region of the TARP protein. As demonstrated using HLA expressing human transgenic mice and C57BL/6 mice, these unique sequences allow for effective antigen processing into MHC Class I and MHS Class II epitopes and presentation of T cell antigens encoded in the TARP protein when delivered in association with cationic lipid nanoparticles.

An important consideration in the design of peptide-based vaccines designed to elicit CD8 T cell and CD4 T cell immune response in humans is the polymorphism of the HLA class I and HLA class II molecules in the population. HLA-Class I can accommodate shot peptides ranging from 8-10 aa long, and the HLA-class II peptides can accommodate peptides sequences ranging from 13-25 amino acids. Because different HLA alleles bind different peptides, it is important that a peptide vaccine contains enough different peptides to be immunogenic in a high percentage of the populations. As demonstrated in the data above, the peptide sequences containing minimal CD8 T cell epitopes or peptide sequences containing multiple CD8 T cell epitopes or with modification to enhance interaction with adjuvant was not sufficient to make the formulation immunogenic. This limitation was overcome by designing long peptides that contain multiple CD4 and CD8 T cell epitopes. The sequences in this unique formulation contain the length of the peptide ranging from 32-38 amino acids long with an overlap of 12 amino acids at the junction. This unique peptide design captures the entire TARP protein sequence to maximize the number of peptides that the formulation can present to the target population. As detailed in the examples below, ELISpot assays and mouse models expressing different MHC class I molecules were used to confirm the presentation, processing, and induction of CD8 T cells specific to TARP protein. Further, peptides that are known to stimulate TARP specific CD8 T cells in an HLA-A2 dependent manner were chosen to confirm the ability to induce human T cell immune response and demonstrated that these long peptide vaccine formulations could be processed to present the verified HLA-A2 specific peptides in HLA-A2 transgenic mice and stimulate peptide specific T cell responses following vaccination as shown in the data provided.

### Multiepitope Peptides

In one embodiment, the present invention provides a multiepitope peptide including at least one multiepitope peptide having an amino acid sequence with at least 80%, 85%, 90% or 95% sequence identity with the amino acid sequence of any one of SEQ ID NOs:27-30 and 32.

The terms “peptide”, “polypeptide” and “protein” are used interchangeably herein and refer to any chain of at least two amino acids, linked by a covalent chemical bound. As used herein polypeptide can refer to the complete amino acid sequence coding for an entire protein or to a portion thereof. A “protein coding sequence” or a sequence that “encodes” a particular polypeptide or peptide, is a nucleic acid sequence that is transcribed (in the case of DNA) and is translated (in the case of mRNA) into a polypeptide in vitro or in vivo when placed under the control of appropriate regulatory sequences. The boundaries of the coding sequence are determined by a start codon at the 5′ (amino) terminus and a translation stop codon at the 3′ (carboxyl) terminus. A coding sequence can include, but is not limited to, cDNA from prokaryotic or eukaryotic mRNA, genomic DNA sequences from prokaryotic or eukaryotic DNA, and even synthetic DNA sequences. A transcription termination sequence will usually be located 3′ to the coding sequence.

The term “long peptide” as used herein refers to a peptide of at least 30 amino acids. The long peptides of the present invention have immunological properties not found in shorter peptides or in pools of shorter peptides that cover the same sequence. Examples of such long peptides include SEQ ID NOs: 27-30 and 32.

In one aspect, the at least one multiepitope peptide has an amino acid sequence with at least 80%, 85%, 90% or 95% sequence identity with any one of SEQ ID NOs:27 and 28. In one aspect, the at least one multiepitope peptide has an amino acid sequence with at least 80%, 85%, 90% or 95% sequence identity with SEQ ID NO:27 and SEQ ID NO:28. In one aspect, the at least one multiepitope peptide has an amino acid sequence with SEQ ID NO:27 and SEQ ID NO:28. In one aspect, the at least one multiepitope peptide having an amino acid sequence of SEQ ID NO:27. In one aspect, the at least one multiepitope peptide having an amino acid sequence of SEQ ID NO: 28.

The terms “sequence identity” or “percent identity” are used interchangeably herein. To determine the percent identity of two polypeptide molecules or two polynucleotide sequences, the sequences are aligned for optimal comparison purposes (e.g., gaps can be introduced in the sequence of a first polypeptide or polynucleotide for optimal alignment with a second polypeptide or polynucleotide sequence). The amino acids or nucleotides at corresponding amino acid or nucleotide positions are then compared. When a position in the first sequence is occupied by the same amino acid or nucleotide as the corresponding position in the second sequence, then the molecules are identical at that position. The percent identity between the two sequences is a function of the number of identical positions shared by the sequences (i.e., % identity=number of identical positions/total number of positions (i.e., overlapping positions)×100). In some embodiments the length of a reference sequence (e.g., any of SEQ ID NOs:1-33) aligned for comparison purposes is at least 80% of the length of the comparison sequence, and in some embodiments is at least 90% or 100%. In an embodiment, the two sequences are the same length.

Ranges of desired degrees of sequence identity are approximately 80% to 100% and integer values in between. Percent identities between a disclosed sequence and a claimed sequence can be at least 80%. Percent identities between a disclosed sequence and a claimed sequence can be at least at least 85%. Percent identities between a disclosed sequence and a claimed sequence can be at least at least 90%. Percent identities between a disclosed sequence and a claimed sequence can be at least at least 95%. Percent identities between a disclosed sequence and a claimed sequence can be at least at least 96%. Percent identities between a disclosed sequence and a claimed sequence can be at least at least 97%. Percent identities between a disclosed sequence and a claimed sequence can be at least at least 98%. Percent identities between a disclosed sequence and a claimed sequence can be at least at least 99%. Percent identities between a disclosed sequence and a claimed sequence can be at least at least 99.5%. Percent identities between a disclosed sequence and a claimed sequence can be at least at least 99.9%. In general, an exact match indicates 100% identity over the length of the reference sequence (e.g., any of SEQ ID NOs: 1-32).

Polypeptides and polynucleotides that are about 85, 86, 87, 88, 89, 90, 91, 92, 93, 94, 95, 96, 97, 98, 99 99.5% or more identical to polypeptides and polynucleotides described herein are embodied within the disclosure. For example, a polypeptide can have 80%, 85%, 90%, 91%, 92%, 93%, 94%, 95%, 96%, 97%, 98%, 99% or more identity to SEQ ID NOs: 2-26, 27, 28, 29, 30, 31 or 32. In some aspects, a polypeptide can have 80% identity to SEQ ID NO:27. In some aspects, a polypeptide can have 80% identity to SEQ ID NO: 27. In some aspects, a polypeptide can have 80% identity to SEQ ID NO:27. In some aspects, a polypeptide can have 85% identity to SEQ ID NO:27. In some aspects, a polypeptide can have 90% identity to SEQ ID NO:27. In some aspects, a polypeptide can have 91% identity to SEQ ID NO:27. In some aspects, a polypeptide can have 92% identity to SEQ ID NO:27. In some aspects, a polypeptide can have 93% identity to SEQ ID NO:27. In some aspects, a polypeptide can have 94% identity to SEQ ID NO:27. In some aspects, a polypeptide can have 95% identity to SEQ ID NO: 27. In some aspects, a polypeptide can have 96% identity to SEQ ID NO:27. In some aspects, a polypeptide can have 97% identity to SEQ ID NO:27. In some aspects, a polypeptide can have 98% identity to SEQ ID NO:27. In some aspects, a polypeptide can have 99% identity to SEQ ID NO:27. In some aspects, a polypeptide can have 80% identity to SEQ ID NO:28. In some aspects, a polypeptide can have 85% identity to SEQ ID NO: 28. In some aspects, a polypeptide can have 90% identity to SEQ ID NO:28. In some aspects, a polypeptide can have 91% identity to SEQ ID NO:28. In some aspects, a polypeptide can have 92% identity to SEQ ID NO:28. In some aspects, a polypeptide can have 93% identity to SEQ ID NO:28. In some aspects, a polypeptide can have 94% identity to SEQ ID NO:28. In some aspects, a polypeptide can have 95% identity to SEQ ID NO:28. In some aspects, a polypeptide can have 96% identity to SEQ ID NO:28. In some aspects, a polypeptide can have 97% identity to SEQ ID NO:28. In some aspects, a polypeptide can have 98% identity to SEQ ID NO:28. In some aspects, a polypeptide can have 99% identity to SEQ ID NO:28.

Variants of the disclosed sequences also include peptides, or full-length protein, that contain substitutions, deletions, or insertions into the protein backbone, that would still leave at least about 70% homology to the original protein over the corresponding portion. A yet greater degree of departure from homology is allowed if like-amino acids, i.e., conservative amino acid substitutions, do not count as a change in the sequence. Examples of conservative substitutions involve amino acids that have the same or similar properties. Illustrative amino acid conservative substitutions include the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine, glutamine, or glutamate; methionine to leucine or isoleucine; phenylalanine to tyrosine, leucine or methionine; serine to threonine; threonine to serine; tryptophan to tyrosine; tyrosine to tryptophan or phenylalanine; valine to isoleucine to leucine.

As used herein, the terms “polyepitope peptide”, “multiepitope peptide” and the like refer to peptide or polypeptide that includes at least two epitopes as described herein. For example, the polyepitope peptide includes 2, 3, 4, 5, 6, 7, 8, 9, 10 or more of the epitopes of the invention.

The term “epitope” refers to an antigenic determinant in a molecule such as an antigen, i.e., to a part in or fragment of the molecule that is recognized by the immune system. An epitope of a protein such as a tumor antigen preferably comprises a continuous or discontinuous portion of said protein. The terms “epitope”, “antigen peptide”, “antigen epitope”, “immunogenic peptide”, “antigenic fragment” and “MHC binding peptide” can be used interchangeably herein and preferably relate to a representation of an antigen which is capable of eliciting an immune response against the antigen or a cell expressing or comprising and preferably presenting the antigen. An “antigen” according to the invention covers any substance that will elicit an immune response. In particular, an “antigen” relates to any substance, preferably a peptide or protein, that reacts specifically with antibodies or T-lymphocytes (T cells). According to the present invention, the term “antigen” comprises any molecule which comprises at least one epitope. Preferably, an antigen in the context of the present invention is a molecule which, optionally after processing, induces an immune reaction. According to the present invention, any suitable antigen may be used, which is a candidate for an immune reaction, wherein the immune reaction is preferably a cellular immune reaction. In the context of the embodiments of the present invention, the antigen is preferably presented by a cell, preferably by an antigen presenting cell which includes a diseased cell, in particular a cancer cell, in the context of MHC molecules, which results in an immune reaction against the antigen. An antigen is preferably a product which corresponds to or is derived from a naturally occurring antigen. Such naturally occurring antigens include tumor antigens.

The epitopes referred to in the present application include any epitope that can be derived from a TARP peptide having the amino acid sequence of SEQ ID NO:1.

Nonlimiting examples of multiepitope peptides include SEQ ID NO:27, SEQ ID NO:28, SEQ ID NO:29, SEQ ID NO:30, and SEQ ID NO:32.

A multiepitope peptide of the present invention, can include at least one multiepitope peptide, at least two multiepitope peptides, at least three multiepitope peptides, at least four multiepitope peptides, at least five multiepitope peptides, at least six multiepitope peptides, at least seven multiepitope peptides, at least eight multiepitope peptides, at least nine multiepitope peptides and at least ten multiepitope peptides. In one aspect the, multiepitope peptide includes at least two multiepitope peptides. A multiepitope peptide of the present invention, can include one multiepitope peptide, two multiepitope peptides, three multiepitope peptides, four multiepitope peptides, five multiepitope peptides, six multiepitope peptides, seven multiepitope peptides, eight multiepitope peptides, nine multiepitope peptides and ten multiepitope peptides. In one aspect the, multiepitope peptide includes two multiepitope peptides.

The multiepitope peptides of the present invention may be modified. In one aspect, the at least one multiepitope peptide is covalently modified. In one aspect, the modification includes palmitoylation or addition of an anionic sequence. In one aspect, the anionic sequence is SSEEEDE (SEQ ID NO:33). In one aspect, the anionic sequence is SSEEEDEE (SEQ ID NO:34). In one aspect, the anionic sequence is SEEEDESS (SEQ ID NO:35). In one aspect, the anionic sequence is SEEEDESEED (SEQ ID NO:36).

### Compositions of Multiepitope Peptides

In one embodiment, the present invention provides a composition including one or more multiepitope peptides having an amino acid sequence with at least 80%, 85%, 90% or 95% sequence identity with the amino acid sequence of any one of SEQ ID NOs:27-30 and 32 and an adjuvant, wherein the adjuvant is a cationic lipid.

As used herein the term composition is meant to include pharmaceutical compositions, which may also contain other therapeutic agents, and may be formulated, for example, by employing conventional pharmaceutically acceptable vehicles or diluents, as well as pharmaceutical additives of a type appropriate to the mode of desired administration (for example, excipients, preservatives, etc.) according to techniques known in the art of pharmaceutical formulation. In certain embodiments, the compositions disclosed herein are formulated with additional agents that promote entry into the desired cell or tissue. Such additional agents include micelles, liposomes, and dendrimers.

The term “pharmaceutically acceptable” refers to the fact that the carrier, diluent or excipient must be compatible with the other ingredients of the formulation and not deleterious to the recipient thereof. For example, the carrier, diluent, or excipient or composition thereof may be administered to a subject along with a conjugate of the invention without causing any undesirable biological effects or interacting in an undesirable manner with any of the other components of the pharmaceutical composition in which it is contained.

Pharmaceutical compositions including the peptides or compositions described herein may be administered by any suitable means, for example, parenterally, such as by subcutaneous, intravenous, intramuscular, intrathecal, or intracisternal injection or infusion techniques (e.g., as sterile injectable aqueous or non-aqueous solutions or suspensions) in dosage formulations containing non-toxic, pharmaceutically acceptable vehicles or diluents. Depending on the condition being treated, these pharmaceutical compositions may be formulated and administered systemically or locally. Techniques for formulation and administration are generally known in the art. Suitable routes may, for example, parenteral delivery, including intramuscular, subcutaneous, intramedullary, intrathecal, intraventricular, intravenous, or intraperitoneal. For injection, the pharmaceutical compositions of the invention may be formulated in aqueous solutions, preferably in physiologically compatible buffers such as water, Hanks' solution, Ringer's solution, or physiologically buffered saline.

Adjuvants are often used to modify or augment the effects of a vaccine by stimulating the immune system to respond to the vaccine more vigorously, and thus providing increased immunity to a particular disease. Adjuvants accomplish this task by mimicking specific sets of evolutionarily conserved molecules, so called pathogen-associated molecular patterns, which include liposomes, lipopolysaccharide, molecular cages for antigens, components of bacterial cell walls, and endocytosed nucleic acids such as RNA, double-stranded RNA, single-stranded DNA, and unmethylated CpG dinucleotide-containing DNA. Because immune systems have evolved to recognize these specific antigenic moieties, the presence of an adjuvant in conjunction with the vaccine can greatly increase the innate immune response to the antigen by augmenting the activities of dendritic cells, lymphocytes, and macrophages by mimicking a natural infection.

The composition described herein can be formulated with a lipid nanoparticle as an adjuvant to enhance the presentation of the antigens to antigen presenting cells, and therefore to increase the immune response induce by the antigens.

In some aspects described herein, the adjuvant is a cationic lipid. As used herein, the term “cationic lipid” refers to any of a number of lipid species which carry a net positive charge at physiological pH or have a protonatable group and are positively charged at pH lower than the pKa.

Suitable cationic lipid according to the present disclosure include, but are not limited to: 3-β[4NIN, 8-diguanidino spermidine)-carbamoyl]cholesterol (BGSC); 3-β[N,N-diguanidinoethyl-aminoethane)-carbamoyl]cholesterol (BGTC); N,N.1N2N3Tetra-methyltetrapalmitylspermine (cellfectin); N-t-butyl-N′-tetradecyl-3-tetradecyl-aminopropion-amidine (CLONfectin); dimethyldioctadecyl ammonium bromide (DDAB); 1,2-dimyristyloxypropyl-3-dimethyl-hydroxy ethyl ammonium bromide (DMRIE); 2,3-dioleoyloxy-N-[2(sperminecarboxamido)ethyl]-N,N-dimethyl-1-p-ropanaminium trifluorocetate) (DOSPA); 1,3-dioleoyloxy-2-(6-carboxyspermyl)-propyl amide (DOSPER); 4-(2,3-bis-palmitoyloxy-propyl)-1-methyl-1H-imidazole (DPIM) N,N,N′,N′-tetramethyl-N,N′-bis(2-hydroxyethyl)-2,3-dioleoyloxy-1,4-butane-diammonium iodide) (Tfx-50); N-1-(2,3-dioleoyloxy) propyl-N,N,N-trimethyl ammonium chloride (DOTMA) or other N—(N,N-1-dialkoxy)-alkyl-N,N,N-trisubstituted ammonium surfactants; 1,2 dioleoyl-3-(4′-trimethylammonio) butanol-sn-glycerol (DOBT) or cholesteryl (4′trimethylammonia) butanoate (ChOTB) where the trimethylammonium group is connected via a butanol spacer arm to either the double chain (for DOTB) or cholesteryl group (for ChOTB); DORI (DL-1,2-dioleoyl-3-dimethylaminopropyl-.beta.-hydroxyethylammonium) or DORIE (DL-1,2-O-dioleoyl-3-dimethylaminopropyl-.beta.-hydroxyethylammonium) (DORIE) or analogs thereof as disclosed in WO 93/03709; 1,2-dioleoyl-3-succinyl-sn-glycerol choline ester (DOSC); cholesteryl hemisuccinate ester (ChOSC); lipopolyamines such as dioctadecylamidoglycylspermine (DOGS) and dipalmitoyl phosphatidylethanolamylspermine (DPPES), cholesteryl-3β-carboxyl-amido-ethylenetrimethylammonium iodide, 1-dimethylamino-3-trimethylammonio-DL-2-propyl-cholesteryl carboxylate iodide, cholesteryl-3-O-carboxyamidoethyleneamine, cholesteryl-3-.beta.-oxysuccinamido-ethylenetrimethylammonium iodide, 1-dimethylamino-3-trimethylammonio-DL-2-propyl-cholesteryl-3-β-oxysu-ccinate iodide, 2-(2-trimethylammonio)-ethylmethylamino ethyl-cholesteryl-3-.beta.-oxysuccinate iodide, 3-β-N—(N′,N′-dimethylaminoethane) carbamoyl cholesterol (DC-chol), and 3-β-N-(polyethyleneimine)-carbamoylcholesterol; O,O′-dimyristyl-N-lysyl aspartate (DMKE); O,O′-dimyristyl-N-lysyl-glutamate (DMKD); 1,2-dimyristyloxypropyl-3-dimethyl-hydroxy ethyl ammonium bromide (DMRIE); 1,2-dilauroyl-sn-glycero-3-ethylphosphocholine (DLEPC); 1,2-dimyristoyl-sn-glycero-3-ethylphosphocholine (DMEPC); 1,2-dioleoyl-sn-glycero-3-ethylphosphocholine (DOEPC); 1,2-dipalmitoyl-sn-glycero-3-ethylphosphocholine (DPEPC); 1,2-distearoyl-sn-glycero-3-ethylphosphocholine (DSEPC); 1,2-dioleoyl-3-trimethylammonium propane (DOTAP); dioleoyl dimethylaminopropane (DODAP); 1,2-palmitoyl-3-trimethylammonium propane (DPTAP); 1,2-distearoyl-3-trimethylammonium propane (DSTAP), 1,2-myristoyl-3-trimethylammonium propane (DMTAP); and sodium dodecyl sulfate (SDS). Furthermore, structural variants and derivatives of the any of the described cationic lipids are also contemplated.

In some aspects, the cationic lipid is DOTAP, DDA, DOEPC, DOTMA, R-DOTAP, R-DDA, R-DOEPC, R-DOTMA, S-DOTAP, S-DDA, S-DOEPC, S-DOTMA, variations thereof, analogs thereof and combinations thereof. In other aspects, the cationic lipid is DOTAP. In yet other aspects, the cationic lipid is DOTMA. In other aspects, the cationic lipid is DOEPC. In some aspects, the cationic lipid is purified.

In some embodiments, the cationic lipid is an enantiomer of a cationic lipid. The term “enantiomer” refers to a stereoisomer of a cationic lipid which is a non-superimposable mirror image of its counterpart stereoisomer, for example R and S enantiomers. In various examples, the enantiomer is R-DOTAP or S-DOTAP. In one example, the enantiomer is R-DOTAP. In another example, the enantiomer is S-DOTAP. In some aspects, the enantiomer is purified.

In one aspect, the cationic lipid is selected from the group consisting of DOTAP, DDA, DOEPC, DOTMA, R-DOTAP, R-DDA, R-DOEPC, R-DOTMA, S-DOTAP, S-DDA, S-DOEPC, S-DOTMA, variations thereof and analogs thereof.

In one aspect, the at least one multiepitope peptide has an amino acid sequence with at least 80%, 85%, 90% or 95% sequence identity with any one of SEQ ID NOs:27 and 28. In one aspect, the at least one multiepitope peptide has an amino acid sequence with at least 80%, 85%, 90% or 95% sequence identity with SEQ ID NO:27 and SEQ ID NO:28. In one aspect, the at least one multiepitope peptide has an amino acid sequence with SEQ ID NO:27 and SEQ ID NO:28. In one aspect, the at least one multiepitope peptide having an amino acid sequence of SEQ ID NO:27. In one aspect, the at least one multiepitope peptide having an amino acid sequence of SEQ ID NO: 28.

In another aspect, the one or more multiepitope peptides are oxidized, cross-linked, pegylated, glycosylated, phosphorylated, palmitoylated, methylated, or biotinylated.

In one aspect, the one or more multiepitope peptides are modified by palmitoylation or by the addition of at least one anionic amino acid.

In one aspect, the one or more multiepitope peptides are encapsulated in liposomes comprising cationic lipid nanoparticles or mixed as separate micelles with preformed cationic lipid nanoparticles.

The peptides/multiepitope peptides described herein are engineered through lipidation to spontaneously form high molecular weight micellar structures. In some aspects, the peptides/multiepitope peptides do not need to interact with the cationic lipid (e.g., R-DOTAP) nanoparticles for form micelles.

In one aspect, the one or more multiepitope peptides and the preformed cationic lipid nanoparticles are mixed at a 1:1 ratio.

In another aspect, the composition further includes an enhancer agonist epitope and/or analogs thereof.

### Vaccine Compositions

In one embodiment, the present invention provides a vaccine composition including at least one multiepitope peptides having at least one multiepitope peptide, wherein the multiepitope peptide includes at least one TARP peptide, and an adjuvant, wherein the adjuvant is a cationic lipid.

According to the invention, the term “vaccine” relates to a pharmaceutical preparation (pharmaceutical composition) or product that upon administration induces an immune response, in particular a cellular immune response, which recognizes and attacks a pathogen or a diseased cell such as a cancer cell. A vaccine may be used for the prevention or treatment of a disease. The term “individualized cancer vaccine” concerns a particular cancer patient and means that a cancer vaccine is adapted to the needs or special circumstances of an individual cancer patient.

By covering “a T-cell receptor alternate reading frame protein (TARP) amino acid sequence” it is meant that the amino acid sequences of the peptides when combined cover the entirety of the amino acid sequence of a TARP protein, for example having the amino acid sequence of SEQ ID NO: 1. The covering may include overlaps between the amino acid sequences of the peptides.

In another aspect, the at least one multiepitope peptide is modified.

In another aspect, the at least one multiepitope peptide is covalently modified.

In some aspects, the modification includes palmitoylation or addition of an anionic sequence.

In one aspect, the at least one multiepitope peptide has an amino acid sequence with at least 80%, 85%, 90% or 95% sequence identity with the amino acid sequence of any one of SEQ ID NOs:27-30 and 32.

In one aspect, the at least one multiepitope peptide has an amino acid sequence with at least 80%, 85%, 90% or 95% sequence identity with any one of SEQ ID NOs:27 and 28.

In one aspect, the at least one multiepitope peptide has an amino acid sequence with at least 80%, 85%, 90% or 95% sequence identity with SEQ ID NO:27 and SEQ ID NO:28.

In one aspect, the at least one multiepitope peptide has an amino acid sequence of SEQ ID NOs:27 and 28.

In one aspect, the at least one multiepitope peptide has an amino acid sequence of SEQ ID NO:27.

In one aspect, the at least one multiepitope peptide has an amino acid sequence of SEQ ID NO: 28.

In one aspect, the at least one TARP peptide includes an amino acid sequence comprising any of SEQ ID NOs:2-8.

In some aspects, the at least one multiepitope peptide is encapsulated with liposomes including cationic lipids or mixed as separate micelles with preformed cationic lipid nanoparticles. In various aspects, the at least one multiepitope peptide and the preformed cationic lipid nanoparticles are mixed at a 1:1 ratio.

In one aspect, the at least one multiepitope peptide cover a T-cell receptor alternate reading frame protein (TARP) amino acid sequence.

In one aspect, the cationic lipid is DOTAP, DDA, DOEPC, DOTMA, R-DOTAP, R-DDA, R-DOEPC, R-DOTMA, S-DOTAP, S-DDA, S-DOEPC, S-DOTMA, variations thereof or analogs thereof. In one aspect, the cationic lipid is R-DOTAP.

In one aspect, the at least one multiepitope peptide binds to CD4+ T cells and/or CD8+ T cells.

### Methods of Treatment

In a further embodiment, the present invention provides a method of treating cancer in a subject including administering to the subject the vaccine composition including at least one multiepitope peptide, wherein the at least one multiepitope peptide includes at least one TARP peptide, and an adjuvant, wherein the adjuvant is a cationic lipid.

The term “subject” as used herein refers to any individual or patient to which the subject methods are performed. Generally, the subject is human, although as will be appreciated by those in the art, the subject may be an animal. Thus, other animals, including vertebrate such as rodents (including mice, rats, hamsters and guinea pigs), cats, dogs, rabbits, farm animals including cows, horses, goats, sheep, pigs, chickens, etc., and primates (including monkeys, chimpanzees, orangutans and gorillas) are included within the definition of subject.

The term “treatment” is used interchangeably herein with the term “therapeutic method” and refers to both 1) therapeutic treatments or measures that cure, slow down, lessen symptoms of, and/or halt progression of a diagnosed pathologic conditions or disorder, and 2) and prophylactic/preventative measures. Those in need of treatment may include individuals already having a particular medical disorder as well as those who may ultimately acquire the disorder (i.e., those needing preventive measures).

The terms “therapeutically effective amount”, “effective dose,” “therapeutically effective dose”, “effective amount,” or the like refer to that amount of the subject compound that will elicit the biological or medical response of a tissue, system, animal or human that is being sought by the researcher, veterinarian, medical doctor or other clinician. Generally, the response is either amelioration of symptoms in a patient or a desired biological outcome (e.g., treating cancer).

The terms “administration of” and or “administering” should be understood to mean providing a pharmaceutical composition in a therapeutically effective amount to the subject in need of treatment. Administration routes can be enteral, topical or parenteral. As such, administration routes include but are not limited to intracutaneous, subcutaneous, intravenous, intraperitoneal, intraarterial, intrathecal, intracapsular, intraorbital, intracardiac, intradermal, transdermal, transtracheal, subcuticular, intraarticulare, subcapsular, subarachnoid, intraspinal and intrasternal, oral, sublingual buccal, rectal, vaginal, nasal ocular administrations, as well infusion, inhalation, and nebulization.

In one aspect, the at least one multiepitope peptide including an amino acid sequence with at least 80%, 85%, 90% or 95% sequence identity with the amino acid sequence of any one of SEQ ID NOs:27-30 and 32.

In one aspect, the at least one multiepitope peptide has an amino acid sequence with at least 80%, 85%, 90% or 95% sequence identity with any one of SEQ ID NOs:27 and 28. In one aspect, the at least one multiepitope peptide has an amino acid sequence with at least 80%, 85%, 90% or 95% sequence identity with SEQ ID NO:27 and SEQ ID NO:28. In one aspect, the at least one multiepitope peptide has an amino acid sequence of SEQ ID NOs:27 and 28. In one aspect, the at least one multiepitope peptide has an amino acid sequence of SEQ ID NO:27. In one aspect, the at least one multiepitope peptide has an amino acid sequence of SEQ ID NO: 28.

In one aspect, the at least one TARP peptide includes an amino acid sequence comprising any of SEQ ID NOs:2-8.

In some aspects, the at least one multiepitope peptide includes a the first multiepitope peptide, a the second multiepitope peptide.

For example, the vaccine can include SEQ ID NO:27 as the first multiepitope peptide, and SEQ ID NO:28 as the second multiepitope peptide. Alternatively, the vaccine composition can include (i) SEQ ID NO:27 as the first multiepitope peptide, and SEQ ID NO:30 as the second multiepitope peptide; (ii) SEQ ID NO:29 as the first multiepitope peptide, and SEQ ID NO:28 as the second multiepitope peptide; (iii) SEQ ID NO:29 as the first multiepitope peptide, and SEQ ID NO:30 as the second multiepitope peptide; (iv) SEQ ID NO:32 as the first multiepitope peptide, and SEQ ID NO:28 as the second multiepitope peptide; or (v) SEQ ID NO:32 as the first multiepitope peptide, and SEQ ID NO:30 as the second multiepitope peptide.

The vaccine can also include more than two multiepitope peptides. Non-limiting examples of such combinations can include: (i) SEQ ID NOs: 27, 28, 29, (ii) SEQ ID NOs:27, 29, 30, (iii) SEQ ID NOs:29, 32, 28, (iv) SEQ ID NOs:29, 32, 30, (v) SEQ ID NOs:27, 32, 28, (vi) SEQ ID NOs:27, 32, 30, (vii) SEQ ID NOs:28, 30, 27, (viii) SEQ ID NOs:28, 30, 29, (ix) SEQ ID NOs:28, 30, 32, (x) SEQ ID NOs:27, 29, 28, 30, (xi) SEQ ID NOs:27, 32, 28, 30, and (xii) SEQ ID NOs:27, 29, 32, 28, 30.

In another aspect, the cationic lipid is selected from the group consisting of DOTAP, DDA, DOEPC, DOTMA, R-DOTAP, R-DDA, R-DOEPC, R-DOTMA, S-DOTAP, S-DDA, S-DOEPC, S-DOTMA, variations thereof and analogs thereof.

In one aspect, the cationic lipid is R-DOTAP.

In another aspect, the first and the second peptides are encapsulated with cationic lipid nanoparticles or mixed as separate micelles with pre-formed cationic lipid nanoparticles.

In another aspect, the first and the second multiepitope peptides and the preformed cationic lipid nanoparticles are mixed at a 1:1 ratio.

In one aspect, the at least one multiepitope peptide cover a T-cell receptor alternate reading frame protein (TARP) amino acid sequence. In one aspect, the at least one multiepitope peptide is modified. In one aspect, the at least one multiepitope peptide is covalently modified. In one aspect, the modification includes palmitoylation or addition of an anionic sequence.

In one aspect, the first and the second peptides induce the presentation of non-HLA restricted peptides to CD4⁺ and CD8⁺ T cells by antigen presenting cells.

The epitopes and multiepitope peptides described herein are HLA-class I and/or HLA-class II non-restricted epitopes.

The human leukocyte antigen (HLA) system or complex is a complex of genes located on chromosome 6 in humans, and which encode cell-surface proteins responsible for the regulation of the immune system. The HLA system is also known as the human version of the major histocompatibility complex (MHC) found in many animals. HLA genes are highly polymorphic, which means that they have many different alleles, allowing them to fine-tune the adaptive immune system. HLAs corresponding to MHC class I (A, B, and C), all of which are the HLA Class1 group, present peptides from inside the cell. These peptides are produced from digested proteins that are broken down in the proteasomes. In general, these particular peptides are small polymers, of about 8-10 amino acids in length. Foreign antigens presented by MHC class I attract T-lymphocytes called killer T-cells (also referred to as CD8-positive or cytotoxic T-cells) that destroy cells. MHC class I proteins associate with B2-microglobulin, which unlike the HLA proteins is encoded by a gene on chromosome 15. HLAs corresponding to MHC class II (DP, DM, DO, DQ, and DR) present antigens from outside of the cell to T-lymphocytes. These particular antigens stimulate the multiplication of T-helper cells (also called CD4-positive T cells), which in turn stimulate antibody-producing B-cells to produce antibodies to that specific antigen. Self-antigens are suppressed by regulatory T cells.

MHC-restricted antigen recognition, MHC restriction or HLA-restriction, refers to the fact that a T cell can interact with a self-major histocompatibility complex molecule and a foreign peptide bound to it but will only respond to the antigen when it is bound to a particular MHC molecule. When foreign proteins enter a cell, they are broken into peptides. These peptides or antigens can derive from pathogens such as viruses or intracellular bacteria. Foreign peptides are brought to the surface of the cell and presented to T cells by proteins called the major histocompatibility complex (MHC). During T cell development, T cells go through a selection process in the thymus to ensure that the T cell receptor (TCR) will not recognize MHC molecule presenting self-antigens, i.e., that its affinity is not too high. High affinity means it will be autoreactive, but no affinity means it will not bind strongly enough to the MHC. The selection process results in developed T cells with specific TCRs that might only respond to certain MHC molecules but not others. The fact that the TCR will recognize only some MHC molecules but not others contribute to “MHC restriction”. The biological reason of MHC restriction is to prevent supernumerary wandering lymphocytes generation, hence energy saving and economy of cell-building materials. T-cells are a type of lymphocyte that is significant in the immune system to activate other immune cells. T-cells will recognize foreign peptides through T-cell receptors (TCRs) on the surface of the T cells, and then perform different roles depending on the type of T cell they are in order to defend the host from the foreign peptide, which may have come from pathogens like bacteria, viruses or parasites. Enforcing the restriction that T cells are activated by peptide antigens only when the antigens are bound to self-MHC molecules, MHC restriction adds another dimension to the specificity of T cell receptors so that an antigen is recognized only as peptide-MHC complexes. MHC restriction in T cells occurs during their development in the thymus, specifically positive selection. Only the thymocytes (developing T cells in the thymus) that are capable of binding, with an appropriate affinity, with the MHC molecules can receive a survival signal and go on to the next level of selection. MHC restriction is significant for T cells to function properly when it leaves the thymus because it allows T cell receptors to bind to MHC and detect cells that are infected by intracellular pathogens, viral proteins and bearing genetic defects.

In some aspects, the epitope peptides include TARP epitopes. Peptide epitopes, such as TARP epitopes can have affinity with different MHC molecules expressed by antigen presenting cells (APCs), which in turn indicates which immune cell receptors can be presented those peptides epitopes, which immune cell receptor recognize it, and therefore which type of immune response can be induced by said peptide epitopes. As detailed in the Examples section the TARP epitopes described herein have affinity for several HLA molecules and are therefore capable of inducing several immune response types.

In some aspects, the MUC1 epitopes have MHC affinity for HLA-A2, A3, All and A24. In various aspects, the MUC1 epitopes are recognized by a CD4+ T cell receptor and/or by a CD8+ T cell receptor.

As used herein, a peptide epitope that is “recognized by” an immune cell receptor can interchangeably be refers to as a “immune cell receptor epitope”. That is, a TARP epitope that is recognized by a CD4+ T cell receptor can be referred to as a CD4+ T cell receptor epitope or to a TARP epitope, depending on the emphasis made on the peptide from which the epitope is derived from (e.g., TARP) or on the immune cell receptor that it can be recognized by (or interact with based on affinity), (e.g., a CD4+ T cell receptor and/or a CD8+ T cell receptor).

By their nature, multiepitope peptides described herein provide not only HLA-A2 antigens, but also HLA-A3, HLA-A11, HLA-A24 etc. antigens.

In another aspect, treating cancer includes preventing progression of cancer in the subject.

Cancer is a group of diseases involving abnormal cell growth with the potential to invade or spread to other parts of the body. In 2015, about 90.5 million people had cancer, about 14.1 million new cases occur a year and it caused about 8.8 million deaths (15.7% of deaths). The most common types of cancer in males are lung cancer, prostate cancer, colorectal cancer and stomach cancer. In females, the most common types are breast cancer, colorectal cancer, lung cancer and cervical cancer.

The term “cancer” refers to a group of diseases characterized by abnormal and uncontrolled cell proliferation starting at one site (primary site) with the potential to invade and to spread to other sites (secondary sites, metastases) which differentiate cancer (malignant tumor) from benign tumor. Virtually all the organs can be affected, leading to more than 100 types of cancer that can affect humans. Cancers can result from many causes including genetic predisposition, viral infection, exposure to ionizing radiation, exposure environmental pollutant, tobacco and or alcohol use, obesity, poor diet, lack of physical activity or any combination thereof.

As used herein, “neoplasm” or “tumor” including grammatical variations thereof, means new and abnormal growth of tissue, which may be benign or cancerous. In a related aspect, the neoplasm is indicative of a neoplastic disease or disorder, including but not limited, to various cancers. For example, such cancers can include prostate, pancreatic, biliary, colon, rectal, liver, kidney, lung, testicular, breast, ovarian, pancreatic, brain, and head and neck cancers, melanoma, sarcoma, multiple myeloma, leukemia, lymphoma, and the like.

Exemplary cancers described by the national cancer institute include: Acute Lymphoblastic Leukemia, Adult; Acute Lymphoblastic Leukemia, Childhood; Acute Myeloid Leukemia, Adult; Adrenocortical Carcinoma; Adrenocortical Carcinoma, Childhood; AIDS-Related Lymphoma; AIDS-Related Malignancies; Anal Cancer; Astrocytoma, Childhood Cerebellar; Astrocytoma, Childhood Cerebral; Bile Duct Cancer, Extrahepatic; Bladder Cancer; Bladder Cancer, Childhood; Bone Cancer, Osteosarcoma/Malignant Fibrous Histiocytoma; Brain Stem Glioma, Childhood; Brain Tumor, Adult; Brain Tumor, Brain Stem Glioma, Childhood; Brain Tumor, Cerebellar Astrocytoma, Childhood; Brain Tumor, Cerebral Astrocytoma/Malignant Glioma, Childhood; Brain Tumor, Ependymoma, Childhood; Brain Tumor, Medulloblastoma, Childhood; Brain Tumor, Supratentorial Primitive Neuroectodermal Tumors, Childhood; Brain Tumor, Visual Pathway and Hypothalamic Glioma, Childhood; Brain Tumor, Childhood (Other); Breast Cancer; Breast Cancer and Pregnancy; Breast Cancer, Childhood; Breast Cancer, Male; Bronchial Adenomas/Carcinoids, Childhood: Carcinoid Tumor, Childhood; Carcinoid Tumor, Gastrointestinal; Carcinoma, Adrenocortical; Carcinoma, Islet Cell; Carcinoma of Unknown Primary; Central Nervous System Lymphoma, Primary; Cerebellar Astrocytoma, Childhood; Cerebral Astrocytoma/Malignant Glioma, Childhood; Cervical Cancer; Childhood Cancers; Chronic Lymphocytic Leukemia; Chronic Myelogenous Leukemia; Chronic Myeloproliferative Disorders; Clear Cell Sarcoma of Tendon Sheaths; Colon Cancer; Colorectal Cancer, Childhood; Cutaneous T-Cell Lymphoma; Endometrial Cancer; Ependymoma, Childhood; Epithelial Cancer, Ovarian; Esophageal Cancer; Esophageal Cancer, Childhood; Ewing's Family of Tumors; Extracranial Germ Cell Tumor, Childhood; Extragonadal Germ Cell Tumor; Extrahepatic Bile Duct Cancer; Eye Cancer, Intraocular Melanoma; Eye Cancer, Retinoblastoma; Gallbladder Cancer; Gastric (Stomach) Cancer; Gastric (Stomach) Cancer, Childhood; Gastrointestinal Carcinoid Tumor; Germ Cell Tumor, Extracranial, Childhood; Germ Cell Tumor, Extragonadal; Germ Cell Tumor, Ovarian; Gestational Trophoblastic Tumor; Glioma. Childhood Brain Stem; Glioma. Childhood Visual Pathway and Hypothalamic; Hairy Cell Leukemia; Head and Neck Cancer; Hepatocellular (Liver) Cancer, Adult (Primary); Hepatocellular (Liver) Cancer, Childhood (Primary); Hodgkin's Lymphoma, Adult; Hodgkin's Lymphoma, Childhood; Hodgkin's Lymphoma During Pregnancy; Hypopharyngeal Cancer; Hypothalamic and Visual Pathway Glioma, Childhood; Intraocular Melanoma; Islet Cell Carcinoma (Endocrine Pancreas); Kaposi's Sarcoma; Kidney Cancer; Laryngeal Cancer; Laryngeal Cancer, Childhood; Leukemia, Acute Lymphoblastic, Adult; Leukemia, Acute Lymphoblastic, Childhood; Leukemia, Acute Myeloid, Adult; Leukemia, Acute Myeloid, Childhood; Leukemia, Chronic Lymphocytic; Leukemia, Chronic Myelogenous; Leukemia, Hairy Cell; Lip and Oral Cavity Cancer; Liver Cancer, Adult (Primary); Liver Cancer, Childhood (Primary); Lung Cancer, Non-Small Cell; Lung Cancer, Small Cell; Lymphoblastic Leukemia, Adult Acute; Lymphoblastic Leukemia, Childhood Acute; Lymphocytic Leukemia, Chronic; Lymphoma, AIDS-Related; Lymphoma, Central Nervous System (Primary); Lymphoma, Cutaneous T-Cell; Lymphoma, Hodgkin's, Adult; Lymphoma, Hodgkin's; Childhood; Lymphoma, Hodgkin's During Pregnancy; Lymphoma, Non-Hodgkin's, Adult; Lymphoma, Non-Hodgkin's, Childhood; Lymphoma, Non-Hodgkin's During Pregnancy; Lymphoma, Primary Central Nervous System; Macroglobulinemia, Waldenstrom's; Male Breast Cancer; Malignant Mesothelioma, Adult; Malignant Mesothelioma, Childhood; Malignant Thymoma; Medulloblastoma, Childhood; Melanoma; Melanoma, Intraocular; Merkel Cell Carcinoma; Mesothelioma, Malignant; Metastatic Squamous Neck Cancer with Occult Primary; Multiple Endocrine Neoplasia Syndrome, Childhood; Multiple Myeloma/Plasma Cell Neoplasm; Mycosis Fungoides; Myelodysplasia Syndromes; Myelogenous Leukemia, Chronic; Myeloid Leukemia, Childhood Acute; Myeloma, Multiple; Myeloproliferative Disorders, Chronic; Nasal Cavity and Paranasal Sinus Cancer; Nasopharyngeal Cancer; Nasopharyngeal Cancer, Childhood; Neuroblastoma; Non-Hodgkin's Lymphoma, Adult; Non-Hodgkin's Lymphoma, Childhood; Non-Hodgkin's Lymphoma During Pregnancy; Non-Small Cell Lung Cancer; Oral Cancer, Childhood; Oral Cavity and Lip Cancer; Oropharyngeal Cancer; Osteosarcoma/Malignant Fibrous Histiocytoma of Bone; Ovarian Cancer, Childhood; Ovarian Epithelial Cancer; Ovarian Germ Cell Tumor; Ovarian Low Malignant Potential Tumor; Pancreatic Cancer; Pancreatic Cancer, Childhood', Pancreatic Cancer, Islet Cell; Paranasal Sinus and Nasal Cavity Cancer; Parathyroid Cancer; Penile Cancer; Pheochromocytoma; Pineal and Supratentorial Primitive Neuroectodermal Tumors, Childhood; Pituitary Tumor; Plasma Cell Neoplasm/Multiple Myeloma; Pleuropulmonary Blastoma; Pregnancy and Breast Cancer; Pregnancy and Hodgkin's Lymphoma; Pregnancy and Non-Hodgkin's Lymphoma; Primary Central Nervous System Lymphoma; Primary Liver Cancer, Adult; Primary Liver Cancer, Childhood; Prostate Cancer; Rectal Cancer; Renal Cell (Kidney) Cancer; Renal Cell Cancer, Childhood; Renal Pelvis and Ureter, Transitional Cell Cancer; Retinoblastoma; Rhabdomyosarcoma, Childhood; Salivary Gland Cancer; Salivary Gland Cancer, Childhood; Sarcoma, Ewing's Family of Tumors; Sarcoma, Kaposi's; Sarcoma (Osteosarcoma Malignant Fibrous Histiocytoma of Bone; Sarcoma, Rhabdomyosarcoma, Childhood; Sarcoma, Soft Tissue, Adult; Sarcoma, Soft Tissue, Childhood; Sezary Syndrome; Skin Cancer; Skin Cancer, Childhood; Skin Cancer (Melanoma); Skin Carcinoma, Merkel Cell; Small Cell Lung Cancer; Small Intestine Cancer; Soft Tissue Sarcoma, Adult; Soft Tissue Sarcoma, Childhood; Squamous Neck Cancer with Occult Primary, Metastatic; Stomach (Gastric) Cancer; Stomach (Gastric) Cancer, Childhood; Supratentorial Primitive Neuroectodermal Tumors, Childhood; T-Cell Lymphoma, Cutaneous; Testicular Cancer; Thymoma, Childhood; Thymoma, Malignant; Thyroid Cancer; Thyroid Cancer, Childhood; Transitional Cell Cancer of the Renal Pelvis and Ureter; Trophoblastic Tumor, Gestational; Unknown Primary Site, Cancer of, Childhood; Unusual Cancers of Childhood; Ureter and Renal Pelvis, Transitional Cell Cancer; Urethral Cancer; Uterine Sarcoma; Vaginal Cancer; Visual Pathway and Hypothalamic Glioma, Childhood; Vulvar Cancer; Waldenstrom's Macro globulinemia; and Wilms' Tumor.

In one aspect, the cancer includes TARP expressing cancer cells.

By “TARP expressing cancer cells” it is meant that the method described herein can be used for the treatment of any cancer that can be characterized by the presence of cancer cells that express TARP.

In some aspect, the cancer is prostate cancer, breast cancer or acute myeloid leukemia (AML).

In another aspect, the method further includes administering to the subject an anti-cancer treatment. As used herein, the term “anti-cancer treatment” is meant to refer to any therapeutic approach that can be used for the treatment of cancer. It includes, but is not limited to surgery, radiotherapy, chemotherapy, immunotherapy, targeted therapy, and any combination thereof.

In some aspects administration can be in combination with one or more additional therapeutic agents. The phrases “combination therapy”, “combined with” and the like refer to the use of more than one medication or treatment simultaneously to increase the response. The compositions of the present invention might for example be used in combination with other drugs or treatment in use to treat cancer. Specifically, the administration of the peptides, multiepitope peptides, or vaccines described herein to a subject can be in combination with another anti-cancer therapy such as an immune checkpoint inhibitor therapy. Such therapies can be administered prior to, simultaneously with, or following administration of the composition of the present invention.

In various aspects, the immunotherapy includes immune checkpoint inhibitor therapy.

“Checkpoint inhibitor therapy” is a form of cancer treatment currently that uses immune checkpoints which affect immune system functioning. Immune checkpoints can be stimulatory or inhibitory. Tumors can use these checkpoints to protect themselves from immune system attacks. Checkpoint therapy can block inhibitory checkpoints, restoring immune system function. Checkpoint proteins include programmed cell death 1 protein (PDCD1, PD-1; also known as CD279) and its ligand, PD-1 ligand 1 (PD-L1, CD274), cytotoxic T-lymphocyte-associated protein 4 (CTLA-4), A2AR (Adenosine A2A receptor), B7-H3 (or CD276), B7-H4 (or VTCN1), BTLA (B and T Lymphocyte Attenuator, or CD272), IDO (Indoleamine 2,3-dioxygenase), KIR (Killer-cell Immunoglobulin-like Receptor), LAG3 (Lymphocyte Activation Gene-3), TIM-3 (T-cell Immunoglobulin domain and Mucin domain 3), and VISTA (V-domain Ig suppressor of T cell activation).

Programmed cell death protein 1, also known as PD-1 and CD279 (cluster of differentiation 279), is a cell surface receptor that plays an important role in down-regulating the immune system and promoting self-tolerance by suppressing T cell inflammatory activity. PD-1 is an immune checkpoint and guards against autoimmunity through a dual mechanism of promoting apoptosis (programmed cell death) in antigen-specific T-cells in lymph nodes while simultaneously reducing apoptosis in regulatory T cells (anti-inflammatory, suppressive T cells).

PD-1 has two ligands, PD-L1 and PD-L2, which are members of the B7 family. PD-L1 protein is upregulated on macrophages and dendritic cells (DC) in response to LPS and GM-CSF treatment, and on T cells and B cells upon TCR and B cell receptor signaling, whereas in resting mice, PD-L1 mRNA can be detected in the heart, lung, thymus, spleen, and kidney. PD-L1 is expressed on almost all murine tumor cell lines, including PA1 myeloma, P815 mastocytoma, and B16 melanoma upon treatment with IFN-γ. PD-L2 expression is more restricted and is expressed mainly by DCs and a few tumor lines.

CTLA4 or CTLA-4 (cytotoxic T-lymphocyte-associated protein 4), also known as CD152 (cluster of differentiation 152), is a protein receptor that, functioning as an immune checkpoint, downregulates immune responses. CTLA4 is constitutively expressed in regulatory T cells but only upregulated in conventional T cells after activation—a phenomenon which is particularly notable in cancers. CTLA4 is a member of the immunoglobulin superfamily that is expressed by activated T cells and transmits an inhibitory signal to T cells. CTLA4 is homologous to the T-cell co-stimulatory protein, CD28, and both molecules bind to CD80 and CD86, also called B7-1 and B7-2 respectively, on antigen-presenting cells. CTLA-4 binds CD80 and CD86 with greater affinity and avidity than CD28 thus enabling it to outcompete CD28 for its ligands. CTLA4 transmits an inhibitory signal to T cells, whereas CD28 transmits a stimulatory signal. CTLA4 is also found in regulatory T cells and contributes to its inhibitory function. T cell activation through the T cell receptor and CD28 leads to increased expression of CTLA-4.

There are several checkpoint inhibitors that are currently used to treat cancer. PD-1 inhibitors include Pembrolizumab (Keytruda) and Nivolumab (Opdivo). PD-L1 inhibitors include Atezolizumab (Tecentriq), Avelumab (Bavencio) and Durvalumab (Imfinzi). CTLA-4 inhibitors include Iplimumab (Yervoy). There are several other checkpoint inhibitors being developed including an anti B7-H3 antibody (MGA271), an anti-KIR antibody (Lirilumab) and an anti-LAG3 antibody (BMS-986016).

In many aspects, the checkpoint inhibitor therapy includes a programmed cell death 1 protein (PD-1) inhibitor, a PD-1 ligand 1 (PD-L1) inhibitor, and/or a cytotoxic T-lymphocyte-associated protein 4 (CTLA-4) inhibitor.

By “targeted therapy”, it is meant any molecularly targeted therapy used to block the growth of cancer cells by interfering with specific targeted molecules needed for carcinogenesis and tumor growth, rather than by simply interfering with all rapidly dividing cells (e.g., with traditional chemotherapy). Biomarkers are usually required to aid the selection of patients who will likely respond to a given targeted therapy. Non-limiting examples of targeted therapy include: tyrosine-kinase inhibitors, small molecules drug conjugates, serine/threonine kinase inhibitors, monoclonal antibodies and histone deacetylase (HDAC) inhibitor.

In other aspects, the targeted therapy comprises a HDAC inhibitor.

Histone deacetylases (HDAC) are a class of enzymes that remove acetyl groups (O═C—CH3) from an ε-N-acetyl lysine amino acid on both histone and non-histone proteins. HDACs allow histones to wrap the DNA more tightly. This is important because DNA is wrapped around histones, and DNA expression is regulated by acetylation and de-acetylation. HDAC's action is opposite to that of histone acetyltransferase. HDAC proteins are now also called lysine deacetylases (KDAC), to describe their function rather than their target, which also includes non-histone proteins. In general, they suppress gene expression.

Histone deacetylase inhibitors (HDAC inhibitors, HDACi, HDIs) are chemical compounds that inhibit histone deacetylases. The “classical” HDIs act exclusively on Class I, II and Class IV HDACs by binding to the zinc-containing catalytic domain of the HDACs. These classical HDIs can be classified into several groupings named according to the chemical moiety that binds to the zinc ion (except cyclic tetrapeptides which bind to the zinc ion with a thiol group). Some examples in decreasing order of the typical zinc binding affinity:
1. hydroxamic acids (or hydroxamates), such as trichostatin A,
2. cyclic tetrapeptides (such as trapoxin B), and the depsipeptides,
3. benzamides,
4. electrophilic ketones, and
5. the aliphatic acid compounds such as phenylbutyrate and valproic acid.

“Second-generation” HDIs include the hydroxamic acids vorinostat (SAHA), belinostat (PXD101), LAQ824, and panobinostat (LBH589); and the benzamides:entinostat (MS-275), tacedinaline (CI994), and mocetinostat (MGCD0103). The sirtuin Class III HDACs are dependent on NAD+ and are, therefore, inhibited by nicotinamide, as well as derivatives of NAD, dihydrocoumarin, naphthopyranone, and 2-hydroxynaphthaldehydes.

Non-limiting examples of HDAC inhibitor include: Istodax (Romidepsin), Zolinza (Vorinostat), Farydak (Panobinostat) and Belodaq (Belinostat).

In one aspect, treating cancer includes inducing a TARP-specific polyfunctional cytolytic T cell response in the subject.

The term “immune response” refers to an integrated bodily response to an antigen and preferably refers to a cellular immune response or a cellular as well as a humoral immune response. The immune response may be protective/preventive/prophylactic and/or therapeutic.

The immune system is a system of biological structures and processes within an organism that protects against disease. This system is a diffuse, complex network of interacting cells, cell products, and cell-forming tissues that protects the body from pathogens and other foreign substances, destroys infected and malignant cells, and removes cellular debris: the system includes the thymus, spleen, lymph nodes and lymph tissue, stem cells, white blood cells, antibodies, and lymphokines. B cells or B lymphocytes are a type of lymphocyte in the humoral immunity of the adaptive immune system and are important for immune surveillance. T cells or T lymphocytes are a type of lymphocyte that plays a central role in cell-mediated immunity. There are two major subtypes of T cells: the killer T cell and the helper T cell. In addition, there are suppressor T cells which have a role in modulating immune response. Killer T cells only recognize antigens coupled to Class I MHC molecules, while helper T cells only recognize antigens coupled to Class II MHC molecules. These two mechanisms of antigen presentation reflect the different roles of the two types of T cell. A third minor subtype are the γδ T cells that recognize intact antigens that are not bound to MHC receptors. In contrast, the B cell antigen-specific receptor is an antibody molecule on the B cell surface and recognizes whole pathogens without any need for antigen processing. Each lineage of B cell expresses a different antibody, so the complete set of B cell antigen receptors represent all the antibodies that the body can manufacture.

A “cellular immune response”, a “cellular response”, a “cellular response against an antigen” or a similar term is meant to include a cellular response directed to cells characterized by presentation of an antigen with class I or class II MHC. The cellular response relates to cells called T cells or T-lymphocytes which act as either “helpers” or “killers”. The helper T cells (also termed CD4+ T cells) play a central role by regulating the immune response and the killer cells (also termed cytotoxic T cells, cytolytic T cells, CD8+ T cells or CTLs) kill diseased cells such as cancer cells, preventing the production of more diseased cells. In preferred embodiments, the present invention involves the stimulation of an anti-tumor CTL response against tumor cells expressing one or more tumor expressed antigens and preferably presenting such tumor expressed antigens with class I MHC.

The terms “immunoreactive cell” “immune cells” or “immune effector cells” in the context of the present invention relate to a cell which exerts effector functions during an immune reaction. An “immunoreactive cell” preferably is capable of binding an antigen or a cell characterized by presentation of an antigen, or an antigen peptide derived from an antigen and mediating an immune response. For example, such cells secrete cytokines and/or chemokines, secrete antibodies, recognize cancerous cells, and optionally eliminate such cells. For example, immunoreactive cells comprise T cells (cytotoxic T cells, helper T cells, tumor infiltrating T cells), B cells, natural killer cells, neutrophils, macrophages, and dendritic cells.

“Inducing an immune response” may mean that there was no immune response against a particular antigen before induction, but it may also mean that there was a certain level of immune response against a particular antigen before induction and after induction said immune response is enhanced. Thus, “inducing an immune response” also includes “enhancing an immune response”. Preferably, after inducing an immune response in a subject, said subject is protected from developing a disease such as a cancer disease or the disease condition is ameliorated by inducing an immune response. For example, an immune response against a tumor expressed antigen may be induced in a patient having a cancer disease or in a subject being at risk of developing a cancer disease. Inducing an immune response in this case may mean that the disease condition of the subject is ameliorated, that the subject does not develop metastases, or that the subject being at risk of developing a cancer disease does not develop a cancer disease.

Numerous combinations of TARP peptide sequences including specific peptide antigens and long overlapping peptide sequences encompassing the entire TARP protein have been demonstrated to be ineffective when combined with or incorporated into cationic lipid nanoparticles. In addition, various TARP peptide antigen modifications designed to promote association and effective responses when combined with R-DOTAP immunostimulatory nanoparticles including peptide lipidation to form high molecular weight micelles and ionic modifications of peptide sequences (which have been demonstrated to result in strong antigen responses for numerous other peptide and protein antigens combined with R-DOTAP nanoparticles) have been shown to be ineffective for TARP derived peptides. The inventors herein have overcome these challenges for the TARP tumor antigen by designing two specific long overlapping peptides covering the entire TARP sequence that uniquely and effectively associate with cationic immunostimulatory nanoparticles, promoting cellular uptake and processing in this context by dendritic cells and generation of high levels of multi-cytokine producing TARP-specific killer CD8 T-cells.

In one embodiment, the present invention provides a method of inducing a TARP-specific polyfunctional cytolytic T cell response in the subject including administering to the subject the vaccine composition including at least one multiepitope peptides including at least one multiepitope peptide including at least one TARP peptide; and an adjuvant, wherein the adjuvant is a cationic lipid. In one aspect, the at least one multiepitope peptide includes an amino acid sequence with at least 80%, 85%, 90% or 95% sequence identity with any one of SEQ ID NOs: 27-30 and 32. In one aspect, the at least one multiepitope peptide includes an amino acid sequence with at least 80%, 85%, 90% or 95% sequence identity with SEQ ID NO:27 and SEQ ID NO:28. In one aspect, the at least one multiepitope peptide includes an amino acid sequence of SEQ ID NOs:27 and 28. In one aspect, the at least one multiepitope peptide includes an amino acid sequence of SEQ ID NO:27. In one aspect, the at least one multiepitope peptide includes an amino acid sequence of SEQ ID NO: 28. In one aspect, the at least one TARP peptide includes an amino acid sequence comprising any of SEQ ID NOs:2-8. In one aspect, the at least one multiepitope peptide covers a T-cell receptor alternate reading frame protein (TARP) amino acid sequence. In one aspect, the at least one multiepitope peptide is modified. In one aspect, the modification comprises palmitoylation or addition of an anionic sequence. In one aspect, the cationic lipid is DOTAP, DDA, DOEPC, DOTMA, R-DOTAP, R-DDA, R-DOEPC, R-DOTMA, S-DOTAP, S-DDA, S-DOEPC, S-DOTMA, variations thereof or analogs thereof. In one aspect, the cationic lipid is R-DOTAP. In one aspect, the at least one multiepitope peptide is encapsulated with cationic lipid nanoparticles or mixed as separate micelles with preformed cationic lipid nanoparticles. In one aspect, wherein the at least one multiepitope peptide and the preformed cationic lipid nanoparticles are mixed at a 1:1 ratio. In one aspect, the at least one multiepitope peptide induces the presentation of non-HLA restricted peptides to CD4⁺ and CD8⁺ T cells by antigen presenting cells.

An additional important consideration in the design of an effective TARP-based immunotherapeutic designed to elicit both high levels of CD4 and CD8 T cell responses in humans is the polymorphisms of the HLA class I and class II molecules in the population. Because different HLA alleles bind different peptides, it is important that protein/peptide-antigen based immunotherapies contain enough unique high affinity HLA-binding epitopes to be immunogenic in a high percentage of the population. In addition, the peptide antigens must be delivered in association with potent immunostimulation capable of promoting effective antigen uptake by dendritic cells, directing peptide antigen processing into both the class I (CD8) and class II (CD4) pathways, and proving correct cytokine activation and signaling to induce large numbers of multiple cytokine-producing effector T-cells. The inventors herein have overcome these challenges for the TARP tumor antigen by designing two specific long overlapping peptides covering the entire TARP sequence that uniquely and effectively associate with R-DOTAP immunostimulatory nanoparticles, which are taken up and processed in this context by dendritic cells and generate high levels of multi-cytokine producing TARP-specific killer CD8 T-cells. Numerous other combinations of TARP peptide sequences including short specific peptide antigens and other long overlapping peptide sequences from the TARP protein have been demonstrated to be ineffective when combined with or incorporated into cationic lipid nanoparticles. In addition, various TARP peptide antigen modifications including peptide lipidation to form high molecular weight micelles and addition of anionic peptide sequences to promote association of peptide with cationic nanoparticles, which have been demonstrated to result in strong antigen responses for other peptide and protein antigens combined with cationic lipid nanoparticles, have been shown to be ineffective for TARP peptides. The unique combination of the two overlapping TARP peptide sequences reported here results in strong antigen-specific immune responses when delivered in association with cationic lipid nanoparticles. The peptide sequences may be incorporated into immunogenic compositions, such as vaccines. The peptide sequences utilized for the resulting compositions have the benefit of inducing T cell immune responses in a non-HLA restricted manner.

The amino acid sequence of TARP (58 AA residues, Isoelectric point ˜12.3) is as follows:

(SEQ ID NO: 1)
MQMFPPSPLFFFLQLLKQSSRRLEHTFVFLRNFSLMLLRGIGKKRRATR
FWDPRRGTP

Potential peptide antigens within the TARP protein sequence: SEQ ID NO:2: FVFLRNFSL=Wild Type (WT) HLA-A*0201-binding peptide TARP 27-35

SEQ ID NO:3: FLRNFSLMV=Epitope Enhanced (EE) HLA-A*0201-binding peptide TARP 29-37-9V These two short, specific peptides were the original epitopes in a TARP HLA-A2 specific vaccine platform formulated with an oil-in-water adjuvant.

Additional epitopes used in a 2nd TARP vaccine platform with an oil-in water adjuvant are as follows:

Five 20-mer peptides overlapping by 10 residues spanning the entire 58 amino acid TARP protein as shown below:

SEQ ID NO: 4
TARP 1-20: MQMFPPSPLFFFLQLLKQSS
SEQ ID NO: 5
TARP 11-30: FFLQLLKQSSRRLEHTFVFL
SEQ ID NO: 6
TARP 21-40: RRLEHTFVFLRNFSLMLLRG
SEQ ID NO: 7
TARP 31-50: RNFSLMLLRGIGKKRRATRF
SEQ ID NO: 8
TARP 41-58: IGKKRRATRFWDPRRGTP

Presented below are examples discussing non-HLA restricted multiepitope TARP vaccine compositions contemplated for the discussed applications. The following examples are provided to further illustrate the embodiments of the present invention but are not intended to limit the scope of the invention. While they are typical of those that might be used, other procedures, methodologies, or techniques known to those skilled in the art may alternatively be used.

### EXAMPLES

### Example 1

### Evaluate SEQ Id Nos:4-8 in a Cationic Lipid-Based T-Cell Vaccine

In order to establish the feasibility of using R-DOTAP immunostimulatory nanoparticles successfully in a TARP prostate cancer therapeutic vaccine, a series of TARP peptides corresponding to SEQ ID NOs:4-8, with modifications that had been determined to be useful in promoting association with or activity with cationic lipid nanoparticles, including palmitoylation and adding an anionic sequence to the N-terminus to promote charge association with the cationic lipid nanoparticles have been generated. The peptides were formulated with R-DOTAP nanoparticles in two ways; as a 1:1 by volume mixtures of peptide antigen and R-DOTAP nanoparticles where the peptides are not encapsulated in the nanoparticles, and as encapsulated peptide antigens in the R-DOTAP nanoparticles. In cases where the modified peptides form high molecular weight micellar structures (i.e., palmitoylated peptides), it was observed that the antigen complexes will be taken up by dendritic cells along with the R-DOTAP nanoparticles because of their size. Further, the highly charged cationic nanoparticles can also bind the antigen micellar structures facilitating uptake of antigen and immunostimulatory nanoparticles together.

TABLE 1
Unmodified TARP antigens

| Peptide | Sequence |
|---|---|
| TARP 1 (27-35) | FVFLRNFSL (SEQ ID NO: 2) |
| TARP 2 (29-37) | FLRNFSLMV (SEQ ID NO: 3) |
| TARP 3 (4-13) | FLPSPLFFFL (SEQ ID NO: 31) |
| TARP 4 (1-20) | MQMFPPSPLFFFLQLLKQSS (SEQ ID NO: 4) |
| TARP 5 (11-30) | FFLQLLKQSSRRLEHTFVFL (SEQ ID NO: 5) |
| TARP 6 (21-40) | RRLEHTFVFLRNFSLMLLRG (SEQ ID NO: 6) |
| TARP 7 (31-50) | RNFSLMLLRGIGKKRRATRF (SEQ ID NO: 7) |
| TARP 8 (41-58) | IGKKRRATRFWDPRRGTP (SEQ ID NO: 8) |

TABLE 2
Modified TARP antigens

| Peptide | Modification | Sequence |
|---|---|---|
| TARP1 | Palmitoyation | KSS-FVFLRNFSL |
| (27-35) | | (SEQ ID NO: 9) |
| TARP 2 | Palmitoyation | KSS-FLRNFSLMV |
| (29-37) | | (SEQ ID NO: 10) |
| TARP 3 | Palmitoyation | KSS-FPPSPLFFFL |
| (4-13) | | (SEQ ID NO: 11) |
| TARP 4 | Palmitoyation | KSS-MQMFPPSPLFFFLQLLKQSS |
| (1-20) | | (SEQ ID NO: 12) |
| TARP 5 | Palmitoyation | KSS-FFLQLLKQSSRRLEHTFVF |
| (11-30) | | (SEQ ID NO: 33) |
| TARP 6 | Palmitoyation | KSS-RRLEHTFVFLRNFSLMLLRG |
| (21-40) | | (SEQ ID NO: 13) |
| TARP 7 | Palmitoyation | KSS-RNFSLMLLRGIGKKRRATRF |
| (31-50) | | (SEQ ID NO: 14) |
| TARP 8 | Palmitoyation | KSS-IGKKRRATRFWDPRRGTP |
| (41-58) | | (SEQ ID NO: 15) |
| TARP 9 | Palmitoyation | KSS-LQLLKQSSRRLEHTFVFLRR |
| (13-32) | | (SEQ ID NO: 16) |

TABLE 3
Anionic sequence tagged TARP antigens are listed below:

| Peptide | Sequence |
|---|---|
| SSEEEDE-TARP 1 (27-35) | SSEEEDE-FVFLRNFSL (SEQ ID NO: 17) |
| SSEEEDE-TARP 2* (29-37) | SSEEEDE-FLRNFSLMV (SEQ ID NO: 18) |
| SSEEEDE TARP 4 (1-20) | SSEEEDE-MQMFPPSPLFFFLQLLKQSS (SEQ ID NO: 19) |
| SSEEEDE-TARP 5 (11-30) | SSEEEDE-FFLQLLKQSSRRLEHTFVFL (SEQ ID NO: 20) |
| SSEEEDE TARP 6 (21-40) | SSEEEDE-RRLEHTFVFLRNFSLMLLRG (SEQ ID NO: 21) |
| SSEEEDE TARP 6* (21-40) | SSEEEDE-RRLEHTFVFLRNFSLMVLRG (SEQ ID NO: 22) |
| SSEEEDE TARP 7 (31-50) | SSEEEDE-RNFSLMLLRGIGKKRRATRF (SEQ ID NO: 23) |
| SSEEEDE TARP 8 (41-58) | SSEEEDE-IGKKRRATRFWDPRRGTP (SEQ ID NO: 24) |
| TARP S1 | SSEEEDEE-HTFVFLRNFSL (SEQ ID NO: 25) |
| TARP S2* (TARP 1 & | SSEEEDE-FVFLRNFSLMV (SEQ ID NO: 26) |
| mutant TARP 2) | |

### Bioactivity Method:

Randomized mice were divided into groups and each mouse was vaccinated on day 0 and day 7. Each mouse was vaccinated subcutaneously with 100 μl of respective formulations on the flanks of a hind limb. One week after the second vaccination spleens were harvested and processed to create single-cell suspensions. The spleen cells were used to detect antigen-specific T cell activity using an ELISPOT assay to measure IFN-γ. Splenocytes were stimulated with a mixture of long peptide sequences spanning TARP protein to identify TARP-specific T cells.

As illustrated in FIG. 5, TARP peptides 29-37 9V (SEQ ID NO:10), 21-40 (SEQ ID NO:6), 21-40 modified by addition of an anionic amino acid group (SEQ ID NO:21), 21-40 (SEQ ID NO:13) modified by palmitoylation and HPV positive control peptide YT-10 formulated in CFA yielded no antigen-specific ELISPOT responses in HLA-A2 transgenic mice. As seen previously, TARP 27-35 (SEQ ID NO:2 without the 9V substitution) was apparently inactive in this mouse model as shown here it did not generate a response when used as a stimulatory peptide for the ELISPOT. Each marker is an individual mouse response. Antigen dose in vaccine formulations: 100 micrograms.

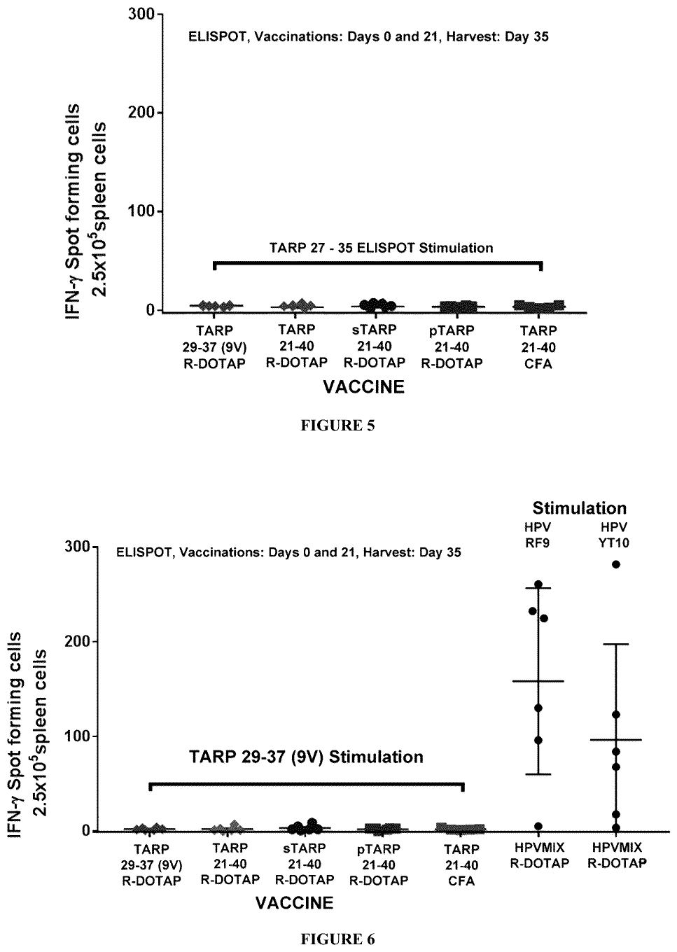

As shown in FIG. 6, TARP peptides 29-37 9V (SEQ ID NO:3), 21-40 (SEQ ID NO:6), 21-40 modified by addition of an anionic amino acid group (SEQ ID NO:21), 21-40 (SEQ ID NO:13) modified by palmitoylation formulated with R-DOTAP nanoparticles and TARP peptide 21-40 (SEQ ID NO:13) formulated in CFA yielded no antigen-specific ELISPOT responses in HLA-A2 transgenic mice. TARP 27-35 (SEQ ID NO:3 with the 9V substitution) was apparently inactive in this mouse model as shown here it did not generate a response when used as a stimulatory peptide for the ELISPOT. Positive control samples of Human Papilloma Virus E7 and E6 oncoprotein peptide antigens formulated with RDOTAP indicated responses observed for two HPV peptide antigens, YT10 and RF9. Each marker was an individual mouse response. Antigen dose in vaccine formulations: 100 micrograms.

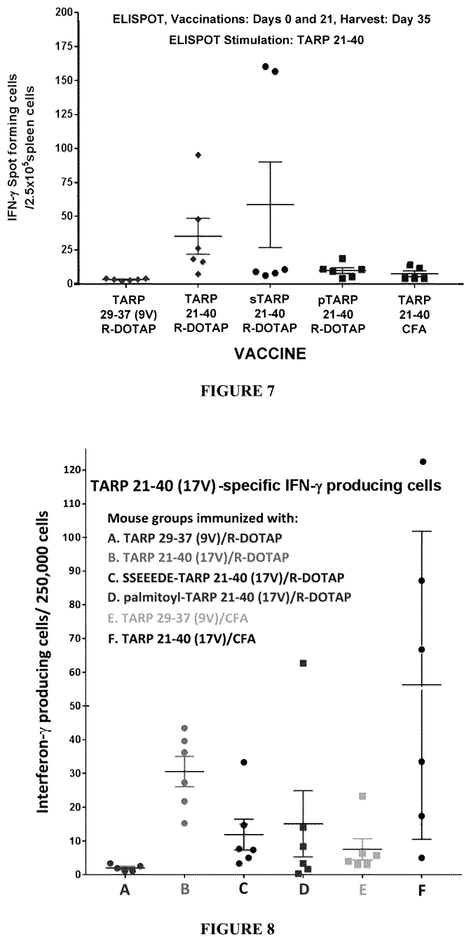

As further shown in FIG. 7, TARP peptides 29-37 9V (SEQ ID NO:3), 21-40 (SEQ ID NO:), 21-40 modified by addition of an anionic amino acid group (SEQ ID NO:21), 21-40 (SEQ ID NO:13) modified by palmitoylation formulated with R-DOTAP nanoparticles and TARP peptide 21-40 (SEQ ID NO: 13) formulated in CFA yielded no or very weak antigen-specific ELISPOT responses in HLA-A2 transgenic mice. TARP peptide antigen 21-40 (SEQ ID NO:6) was inactive or very weakly active in this mouse model as it did not generate a response when used as a stimulatory peptide for the ELISPOT. Peptide 21-40 modified by addition of an anionic amino acid group (SEQ ID NO:21) showed only two of six mice responding to vaccination. Each marker is an individual mouse response. Antigen dose in vaccine formulations: 100 micrograms.

As illustrated in FIG. 8, TARP peptides 29-37 9V (SEQ ID NO:3), 21-40 (SEQ ID NO:6), 21-40 modified by addition of an anionic amino acid group (SEQ ID NO:21), 21-40 (SEQ ID NO:13) modified by palmitoylation formulated with R-DOTAP nanoparticles elicited no or very weak TARP 21-40 ELISPOT responses in our HLA-A2 transgenic mice after two immunizations. In the presence of strong immunoadjuvant Complete Freunds Adjuvant (CFA) TARP 21-40 (17V) (SEQ ID NO:6) was very weakly active when stimulated with TARP 21-40 (SEQ ID NO:6). x-axis: peptide vaccine mixtures. Stimulatory peptide TARP 21-40 (17V) solubilized in water/sucrose in the ELISPOT assay.

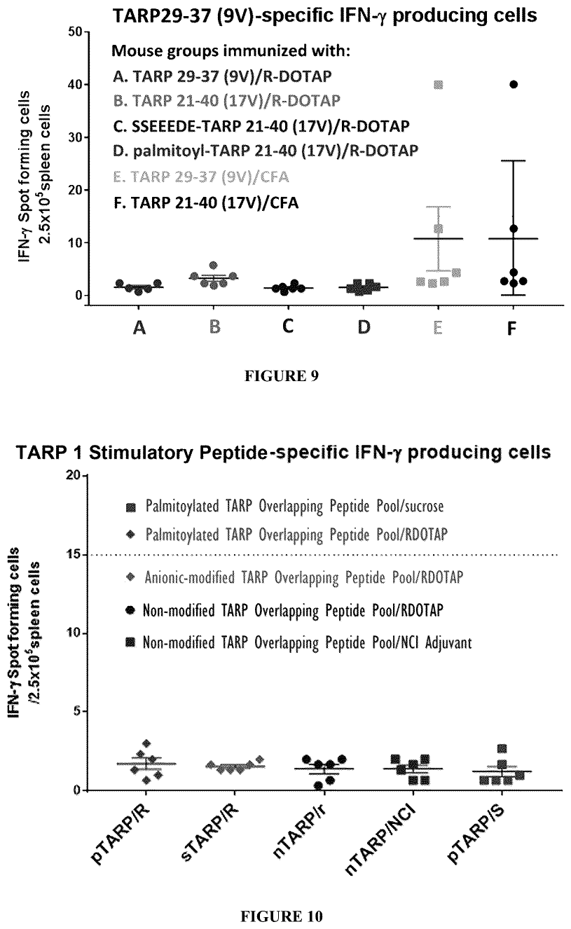

As illustrated in FIG. 9, TARP 29-37 (9V) (SEQ ID NO:3) and TARP 21-40 (17V) (SEQ ID NO:6) or variants of this peptide, when encapsulated in R-DOTAP nanoparticles, did not appear to elicit any TARP 29-37 (9V) ELISPOT responses in our HLA-A2 transgenic mice after two immunizations. Very weak or no ELISPOT responses to TARP 29-37 (9V) (SEQ ID NO:3) were observed when TARP 29-37 (9V) was used to immunize mice in the presence of strong adjuvant Complete Freund's Adjuvant (CFA). x-axis: peptide vaccine mixtures. Stimulatory peptide TARP 29-37 (9V) solubilized in water/sucrose was utilized in the ELISPOT assay (see FIG. 9).

### Example 2

### Development of a Long Multi-Epitope Tarp Peptide T-Cell Vaccine

A novel approach to the TARP program was developed resulting in the development of two long novel peptides including an N-terminal and a C-terminal TARP peptide of 32-38 amino acids that share a 12 aa overlap and also includes at the C-terminus and N-terminus of each peptide respectively an immunogenic region of the TARP protein. The two long overlapping peptides encompass all of the TARP antigens were prepared as follows: the first with the N-terminal amino acid through the previously identified HLA-A2 antigen FLRNFSLMV TARP 29-37-9V (TARP 2, SEQ ID NO:10) (SEQ ID NO:27), and the second with the C-terminal region extending through the same antigen, FLRNFSLMV TARP 29-37-9V (TARP 2, SEQ ID NO:10) (SEQ ID NO:28). Different versions of the long overlapping peptides were also produced to assess whether charge modification by the addition of anionic amino acid groups to the long antigen-overlapping peptides to the peptide terminus would increase association of the antigens with the cationic R-DOTAP nanoparticles, thus resulting in improved immunoactivity.

TARP full length sequence:
(SEQ ID NO: 1)
MQMFPPSPLFFFLQLLKQSSRRLEHTFVFLRNFSLMLLRGIGKKRRATR
FWDPRRGTP
(SEQ ID NO: 27)
MQMFPPSPLFFFLQLLKQSSRRLEHTFVFLRNFSLMVL
(SEQ ID NO: 28)
FVFLRNFSLMVLRGIGKKRRATRFWDPRRGTP
Anionic tag variants:
(SEQ ID NO: 29)
SEEEDESSMQMFPPSPLFFFLQLLKQSSRRLEHTFVFLRNFSLMV
(SEQ ID NO: 30)
SEEEDESEEDFVFLRNFSLMVLRGIGKKRRATRFWDPRRGTP
HLA-A2 antigen sequences:
SEQ ID NO: 2
FVFLRNFSL TARP 27-35 (TARP 1)
SEQ ID NO: 10
FLRNFSLMV TARP 29-37-9V (TARP 2)

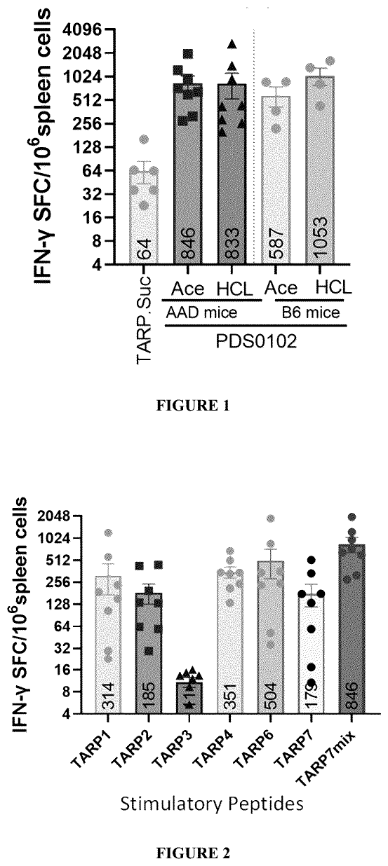

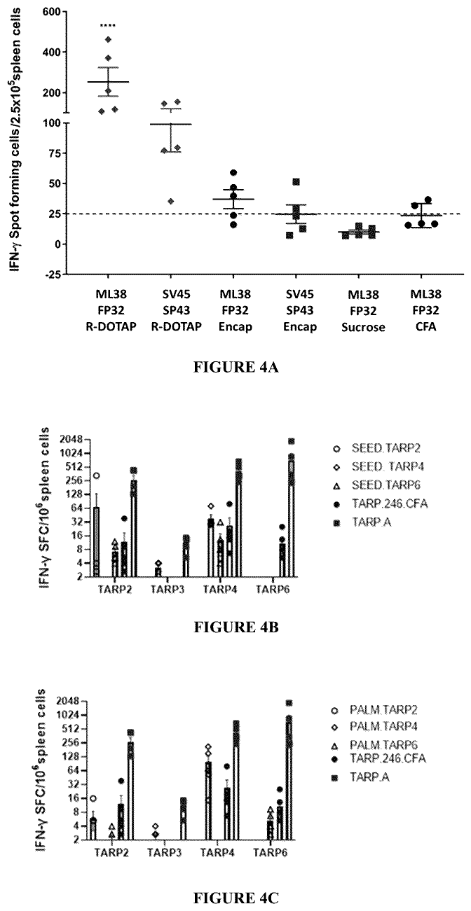

The long peptides were formulated with R-DOTAP by mixing the solubilized peptides with the pre-formed nanoparticles prior to immunization. The formulations were tested in the same murine assays as described in Example 1. The long peptides are formulated at 4 mg/mL each (for FP32 and ML38); R-DOTAP at 6 mg/mL; they are mixed together, so the TARP peptides are at 2 mg/mL with R-DOTAP at 3 mg/mL. The human dose is 1 mL, whereas the dose for mice is 1/10th of the human. The peptides are not encapsulated but are present separately as a mixture with R-DOTAP. The results are shown in FIGS. 1 and 4A and are described below.

Table 2 summarizes the peptide sequences used for these experiments.

TABLE 2
Peptide Sequences:

| SEQ ID NO: | Modification | Sequence |
|---|---|---|
| SEQ ID NO: 1 | None | MQMFPPSPLFFFLQLLKQSSRRLEHTFVFLRNFS |
| | | LMLLRGIGKKRRATRFWDPRRGTP |
| SEQ ID NO: 2 | None | FVFLRNFSL |
| SEQ ID NO: 3 | None | FLRNFSLMV |
| SEQ ID NO: 4 | None | MQMFPPSPLFFFLQLLKQSS |
| SEQ ID NO: 5 | None | FFLQLLKQSSRRLEHTFVFL |
| SEQ ID NO: 6 | None | RRLEHTFVFLRNFSLMLLRG |
| SEQ ID NO: 7 | None | RNFSLMLLRGIGKKRRATRF |
| SEQ ID NO: 8 | None | IGKKRRATRFWDPRRGTP |
| SEQ ID NO: 9 | Palmitoylation | KSS-FVFLRNFSL |
| SEQ ID NO: 10 | Palmitoylation | KSS-FLRNFSLMV |
| SEQ ID NO: 11 | Palmitoylation | KSS-FPPSPLFFFL |
| SEQ ID NO: 12 | Palmitoylation | KSS-MQMFPPSPLFFFLQLLKQSS |
| SEQ ID NO: 13 | Palmitoylation | KSS-RRLEHTFVFLRNFSLMLLRG |
| SEQ ID NO: 14 | Palmitoylation | KSS-RNFSLMLLRGIGKKRRATRF |
| SEQ ID NO: 15 | Palmitoylation | KSS-IGKKRRATRFWDPRRGTP |
| SEQ ID NO: 16 | Palmitoylation | KSS-LQLLKQSSRRLEHTFVFLRR |
| SEQ ID NO: 17 | anionic amino acids | SSEEEDE-FVFLRNFSL |
| SEQ ID NO: 18 | anionic amino acids | SSEEEDE-FLRNFSLMV |
| SEQ ID NO: 19 | anionic amino acids | SSEEEDE-MQMFPPSPLFFFLQLLKQSS |
| SEQ ID NO: 20 | anionic amino acids | SSEEEDE-FFLQLLKQSSRRLEHTFVFL |
| SEQ ID NO: 21 | anionic amino acids | SSEEEDE-RRLEHTFVFLRNFSLMLLRG |
| SEQ ID NO: 22 | anionic amino acids | SSEEEDE-RRLEHTFVFLRNFSLMVLRG |
| SEQ ID NO: 23 | anionic amino acids | SSEEEDE-RNFSLMLLRGIGKKRRATRF |
| SEQ ID NO: 24 | anionic amino acids | SSEEEDE-IGKKRRATRFWDPRRGTP |
| SEQ ID NO: 25 | anionic amino acids | SSEEEDEE-HTFVFLRNFSL |
| SEQ ID NO: 26 | anionic amino acids | SSEEEDE-FVFLRNFSLMV |
| SEQ ID NO: 27 | HLA-A2 antigen | MQMFPPSPLFFFLQLLKQSSRRLEHTFVFLRNFS |
| | sequence | LMVL |
| SEQ ID NO: 28 | HLA-A2 antigen | FVFLRNFSLMVLRGIGKKRRATRFWDPRRGTP |
| | sequence | |
| SEQ ID NO: 29 | anionic amino acids | SEEEDESS-MQMFPPSPLFFFLQLLKQSSRRLEH |
| | | TFVFLRNFSLMV |
| SEQ ID NO: 30 | anionic amino acids | SEEEDESEED- |
| | | FVFLRNFSLMVLRGIGKKRRATRFWDPRRGTP |
| SEQ ID NO: 31 | anionic amino acids | FLPSPLFFFL |
| SEQ ID NO: 32 | anionic amino acids | SEEEDESS-MQMFPPSPLFFFLQLLKQSSRRLEH |
| | | TFVFLRNFSLMLV |
| SEQ ID NO: 33 | | SSEEEDE |
| SEQ ID NO: 34 | | SSEEEDEE |
| SEQ ID NO: 35 | | SEEEDESS |
| SEQ ID NO: 36 | | SEEEDESEED |

### Example 3

Immunological Activity of Long Overlapping Tarp Peptides when Combined with R-DOTAP

Two immunogenic vaccine formulations each consisting of 2 mg each of the two long TARP peptide sequences TARP 1-38 (SEQ ID NO:27) and TARP 27-58 (SEQ ID NO:28) were formulated with 3 mg of R-DOTAP nanoparticles in a 1 ml formulation. The efficacy of these vaccine formulations in generating T cell responses was measured by dosing HLA-A2 expressing transgenic mice (AAD mice) and C57BL/6 mice (B6 mice) with two 0.1 ml subcutaneous injections of the vaccine formulations on day 0 and day 7. Immune responses specific to vaccine formulation were measured in an ELISPOT assay by enumerating TARP antigen-specific T cells, in triplicate wells, in the spleens obtained from vaccinated mice (FIG. 1). Each data point in the graph represents the mean SFU per million splenocytes in vaccinated mice. Error bars represent mean±SEM for five mice in each group. Splenocytes were stimulated with a pooled mixture of peptide sequences (SEQ ID NOs:9, 10, 4, 6, 7, 8, and 5) spanning the TARP protein to identify TARP-specific T cells. This data demonstrates that the two long overlapping sequences (SEQ ID NOs:27 and 28) produce effective antigen processing and presentation enabling the TARP vaccine to generate TARP-specific T-cells in a non-HLA restricted manner.

### Example 4

Tarp Antigen Processing and Induction of Various CD8⁺ and CD4⁺ Tarp Epitopes

An immunogenic vaccine formulation was prepared using of 2 mg each of the two long TARP peptide sequences (SEQ ID NOs:27 and 28) formulated with 3 mg of R-DOTAP nanoparticles in a 1 ml formulation. The efficacy of this vaccine formulation in generating T cell responses was measured by dosing HLA-A2 expressing transgenic mice (AAD mice) with two 0.1 ml subcutaneous injections of the vaccine formulation on day 0 and day 7. Immune responses specific to individual regions of the TARP protein were measured in an ELISPOT assay by enumerating TARP antigen-specific T cells, in triplicate wells, in the spleens obtained from vaccinated mice (FIG. 2). Each data point in the graph represents the mean SFU per million splenocytes in vaccinated mice. Error bars represent mean±SEM for five mice in each group. Splenocytes were stimulated with individual peptide sequences (TARP 1: SEQ ID NO:2; TARP 2: SEQ ID NO:3; TARP3: SEQ ID NO:31; TARP 4: SEQ ID NO:4; TARP 6: SEQ ID NO:6; TARP 7: SEQ ID NO:7; and TARP 7 mix (SEQ ID NOs:2, 3, 31, 4, 6, and 7) which are known to be immunogenic in the context of HLA-A2 to identify TARP specific T cells. This data demonstrates that the TARP vaccine composed of SEQ ID NOs:27 and 28 was effectively processed and presented to multiple known HLA-A2 epitopes in vivo.

### Example 5

Long Tarp Peptides Formulated with R-DOTAP Induce an Enhanced Immune Response without Sequence Modification

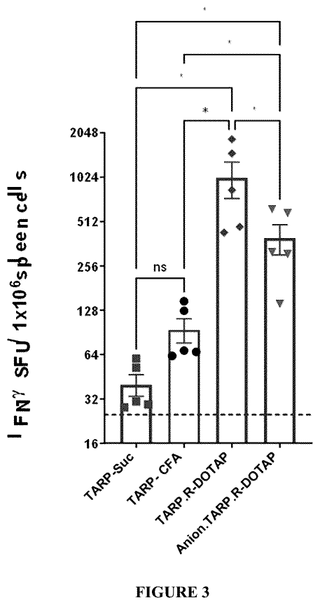

Two immunogenic vaccine formulations were prepared with 2 mg each of two long modified TARP peptide sequences SEQ ID NOs:27 and 28; and SEQ ID NOs:29 and 30) and 3 mg of R-DOTAP in a 1 ml formulation. The efficacy of these vaccine formulations in generating T cell responses was measured by dosing HLA-A2 expressing transgenic mice (AAD mice) with two 0.1 ml subcutaneous injections of the vaccine formulations delivered subcutaneously on day 0 and day 7. SEQ ID NO:29 and SEQ ID NO:30 (contains an additional C-terminal Leucine) are anionic version of the SEQ ID NOs: 27 and 28, respectively, were examined to determine whether charge modification by the addition of the anionic amino acid groups would increase association of the antigens with the cationic R-DOTAP nanoparticles. An additional immunogenic vaccine formulation was prepared with 2 mg each of two long modified TARP peptide sequences SEQ ID NOs:27 and 28) and of Complete Freunds's Adjuvant (CFA) in a formulation. Immune responses specific to the vaccine formulations were measured in an ELISPOT assay by enumerating TARP antigen-specific T cells, in triplicate wells, in the spleens obtained from vaccinated mice (FIG. 3). Each data point in the graph represents the mean SFU per million splenocytes in vaccinated mice. Error bars represent mean±SEM for five mice in each group. Splenocytes were stimulated with a mixture of peptide sequences (SEQ ID NOs:2, 3, 31, 4, 6, and 7) spanning TARP protein to identify TARP-specific T cells. This data demonstrates activity of TARP peptide sequences SEQ ID NOs:27 and 28 when administered in a cationic lipid formulation. About a 50% drop in T-cell induction is seen when the peptides are anion tagged. Additionally, very weak activity was demonstrated when the peptides SEQ ID NOS:27 and 28 are formulated with the potent adjuvant CFA compared with R-DOTAP.

### Example 6

Study Demonstrating that Long Peptides SEQ Id No:27 and 28 Induce an Enhanced Immune Response with R-DOTAP

As illustrated in FIGS. 4A-4C a mixture of long TARP peptides with R-DOTAP nanoparticles induced a strong response in ELISPOT testing. Vaccine formulations were prepared with 2 mg each of two long modified TARP peptide sequences (SEQ ID NOs:27 and 28; and SEQ ID NOs:29 and 30) and 3 mg of R-DOTAP in a 1 ml formulation. The efficacy of these vaccine formulations in generating T cell responses was measured by dosing HLA-A2 expressing transgenic mice (AAD mice) with two 0.1 ml subcutaneous injections of the vaccine formulations delivered subcutaneously on day 0 and day 7 (FIG. 4A). FIGS. 4B-4C provide a graphs showing an example of vaccine formulations consisting of modified short TARP peptide sequences (1-2 mg per peptide) with anion tag (SEED TARP2: SEQ ID NO:18, SEED TARP4: SEQ ID NO:19, SEED TARP6: SEQ ID NO:21,) or lipidated (PALM.TARP2: SEQ ID NO:10, PALM.TARP4: SEQ ID NO:12, PALM.TARP6: SEQ ID NO:13) and formulated with 4-5 mg of R-DOTAP in a 1 ml formulation or formulations consisting 2 mg each of the two long TARP peptide sequences (SEQ ID NO:27) and (SEQ ID NO:28) formulated with 3 mg of R-DOTAP in a 1 ml formulation. Efficacy of these vaccine formulations in generating T cell responses was measured by dosing HLA-A2 expressing transgenic mice (AAD mice) with two 0.1 ml injections of the vaccine delivered subcutaneously on day 0 and day 7. Immune responses specific to vaccine formulation were measured in an ELISPOT assay by enumerating TARP antigen-specific T cells, in triplicate wells, in the spleens obtained from vaccinated mice. TARP246. CFA mice were vaccinated. with TARP peptides in complete Freund's adjuvant. Each data point in the graph represents the mean SFU per million splenocytes in vaccinated mice. Error bars represent mean±SEM for five mice in each group. For identifying TARP-specific T cells, splenocytes were stimulated with verified HLA-A1 binding epitopes TARP2 (SEQ ID NO:4), TARP3 (SEQ ID NO:10), TARP 4 (SEQ ID NO:5), or TARP 6 (SEQ ID NO:6). Peptide encapsulation (Encap) within the R-DOTAP nanoparticles has little effect on ELISPOT responses. TARP peptides modified with an anionic peptide sequence are positive, but non-modified peptides are stronger stimulators. There was no response to TARP peptides noted with sucrose and with the potent immunoadjuvant Complete Freund's Adjuvant (CFA).

### Example 7

Peptide Vaccine Formulations that Fail to Induce an Enhanced Immune Response with R-DOTAP

Vaccine formulations were prepared as shown in Table 4 and evaluated for the ability to induce an enhanced immune response with R-DOTAP, Strong NCI adjuvant (GM-CSF, IL-12, HBV core peptide in IFA) or sucrose. The various vaccine formulations were tested to evaluate long peptides that were modified (i.e., palmitoylated or anion tagged) either admixed with R-DOTAP or encapsulated within R-DOTAP nanoparticles.

TABLE 4
Vaccine Formulations

| | | Number |
|---|---|---|
| Group | Description | of mice |
| Group 1 | pTARPmix-10-AD mix 1:1 by vol. w/6 mg/ml | 6 |
| | RDOTAP | |
| Group 2 | sTARPmix-10-EN no additional RDOTAP needed | 6 |
| Group 3 | nTARPmix-10-EN no additional RDOTAP needed | 6 |
| Group 4 | nTARPmix-10 + NCI adjuvant | 6 |
| Group 5 | pTARPmix-10-AD + Sucrose | 6 |
| | Total number of mice | 30 |

pTARP1-7 peptide pool mixture modified by palmitoylation (SEQ ID NOs: 9-15 and)
sTARP1-7 peptide pool mixture unmodified (SEQ ID NOs: 2-8, and 31)
nTARP1-7 peptide pool mixture modified by anion tag (SEQ ID NOs: 17-24)
EN: encapsulated formulation
AD: admixture formulation

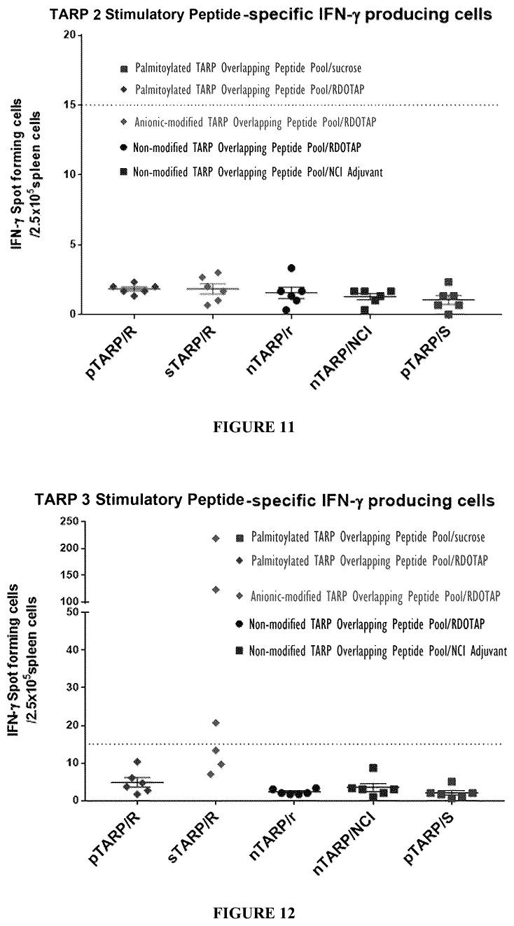

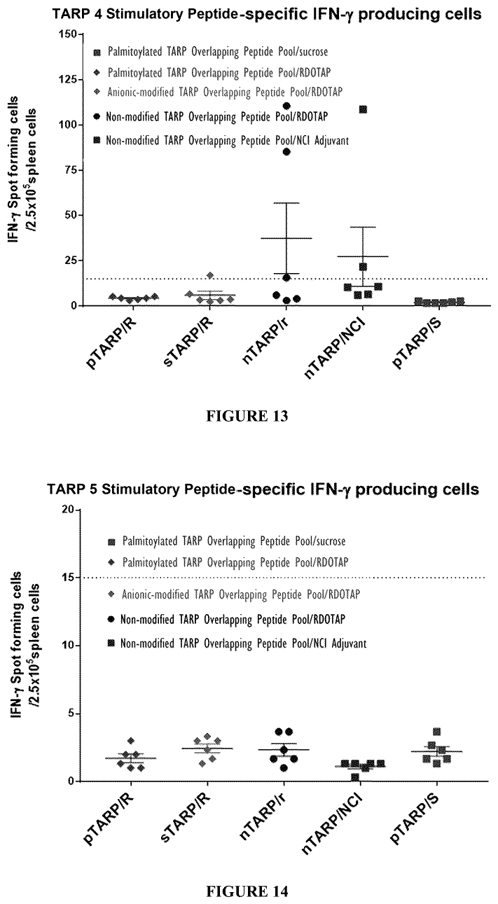

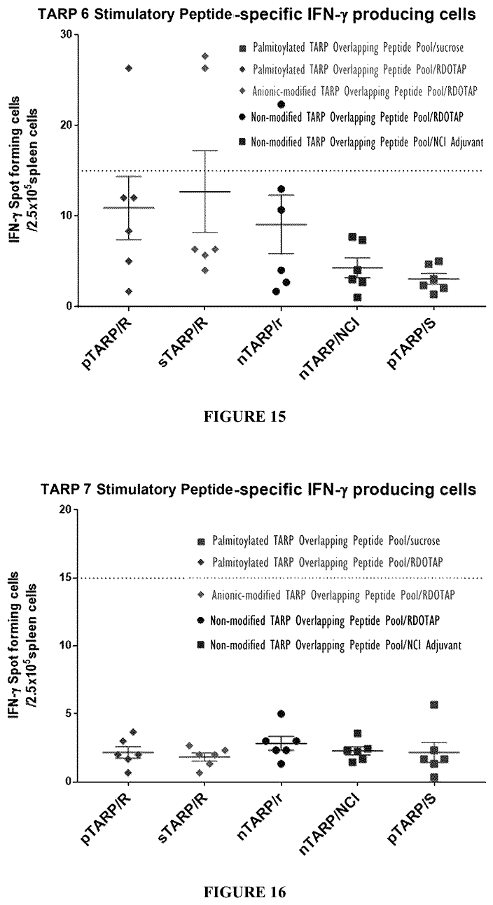

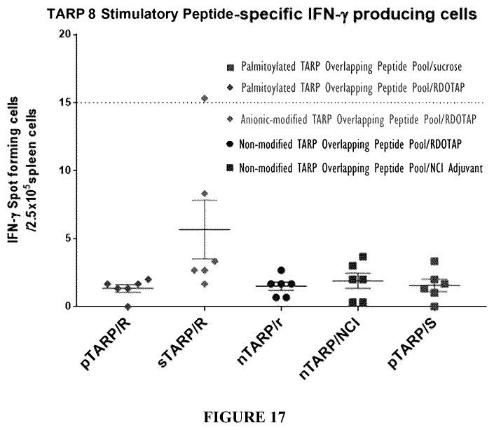

TARP 20-mer overlapping peptide pools, unmodified, palmitoylated or anion tagged, formulated with R-DOTAP, sucrose or strong NCI adjuvant did not induce antigen-specific T-cell responses to TARP peptides 1-8 (SEQ ID NOs:2-8 and 31). TARP 20-mer overlapping peptide pools covering the entire sequence of the protein, including palmitoylated peptide pools and anion-modified peptide pool versions of these peptides were encapsulated in R-DOTAP nanoparticles or admixed with pre-formed R-DOTAP nanoparticles and used to vaccinate HLA-A2 humanized transgenic mice on days 0 and 14. Mouse splenocytes were harvested on day 21 and analyzed for Tarp peptide antigen specific T-cells by ELISPOT (FIGS. 10-17). Mixtures of SEQ ID NOs:2-8, and 31, and their anionic tagged (SEQ ID NOs: 17-24) and palmitoylated derivatives (SEQ ID NOs: 9-15) do not elicit any TARP 1-8 peptide antigens (SEQ ID NOs:2-8 and 31) by ELISPOT responses in HLA-A2 transgenic mice after two immunizations. None of the signals were considered significant compared to minimal baseline responses. Each marker is the mean ELISPOT count of an individual mouse.

The data demonstrate the absence of or very weak immune responses in HLA-A2 transgenic mice to pools of overlapping TARP peptides that span the length of the TARP amino acid sequence is in contrast to the immune response generated by the two long peptides (SEQ ID NOs:27 and 28). These data indicate the two long peptides have unexpected immune properties.

### Example 8

### Overall Conclusion

Disclosed herein are methods for the design and use of unique long peptide sequences derived from TARP protein designed to be effectively processed and presented to T cells in a non-HLA restricted manner when delivered in association with R-DOTAP immunostimulatory nanoparticles, as evidenced by data in FIGS. 1-6. This novel combination contains two long peptide sequences consisting of 32-38 amino acids each. These unique peptide sequences may be incorporated into immunogenic compositions such as vaccines. As demonstrated in FIGS. 1-6, these formulations can induce robust T cell immune responses specific to TARP protein.

Although the invention has been described with reference to the above examples, it will be understood that modifications and variations are encompassed within the spirit and scope of the invention. Accordingly, the invention is limited only by the following claims.

1. A multiepitope peptide comprising at least one multiepitope peptide comprising an amino acid sequence with at least 80% sequence identity with the amino acid sequence of any one of SEQ ID NOs:27-30 and 32.

2-6. (canceled)

7. The multiepitope peptide of claim 1, wherein the at least one multiepitope peptide is modified by palmitoylation or by addition of an anionic sequence.

8. (canceled)

9. A composition comprising one or more multiepitope peptides and an adjuvant,
wherein the adjuvant is a cationic lipid, and wherein the one or more multiepitope peptides comprise at least one TCR gamma alternate reading frame protein (TARP) peptide.

10. The composition of claim 9, wherein the at least one TARP peptide comprises an amino acid sequence with at least 80% sequence identity with the amino acid sequence of any one of SEQ ID NOs:27-30 and 32.

11-15. (canceled)

16. The composition of claim 9, wherein the at least one TARP peptide comprises an amino acid sequence comprising any of SEQ ID NOs:2-8.

17. The composition of claim 9, wherein the cationic lipid is DOTAP, DDA, DOEPC, DOTMA, R-DOTAP, R-DDA, R-DOEPC, R-DOTMA, S-DOTAP, S-DDA, S-DOEPC, S-DOTMA, variations thereof or analogs thereof.

18. (canceled)

19. The composition of claim 9, wherein the one or more multiepitope peptides are modified by palmitoylation or by the addition of at least one anionic amino acid.

20. The composition of claim 9, wherein the one or more multiepitope peptides are encapsulated within cationic liposomes or mixed as separate micelles with preformed cationic lipid nanoparticles.

21. The composition of claim 20, wherein the one or more multiepitope peptides and the preformed cationic lipid nanoparticles are mixed at a 1:1 ratio.

22. The composition of claim 9, further comprising an enhancer agonist epitope and/or an analog thereof.

23. A vaccine composition comprising:
(a) a multiepitope peptide comprising at least one TCR gamma alternate reading frame protein (TARP peptide); and
(b) an adjuvant, wherein the adjuvant is a cationic lipid.

24. The vaccine composition of claim 23, wherein the at least one multiepitope peptide comprises an amino acid sequence with at least 80% sequence identity with any one of SEQ ID NOs:27-30 and 32.

25-28. (canceled)

29. The vaccine composition of claim 23, wherein the at least one TARP peptide comprises an amino acid sequence comprising any of SEQ ID NOs:2-8.

30. The vaccine composition of claim 23, wherein the at least one multiepitope peptide is modified by palmitoylation or by the addition of at least one anionic amino acid.

31-34. (canceled)

35. The vaccine composition of claim 23, wherein the cationic lipid is DOTAP, DDA, DOEPC, DOTMA, R-DOTAP, R-DDA, R-DOEPC, R-DOTMA, S-DOTAP, S-DDA, S-DOEPC, S-DOTMA, variations thereof or analogs thereof.

36. (canceled)

37. The vaccine composition of claim 23, wherein the at least one multiepitope peptide is encapsulated in a cationic liposome or mixed as separate micelles with preformed cationic lipid nanoparticles.

38. The vaccine composition of claim 37, wherein the at least one multiepitope peptide and the preformed cationic lipid nanoparticles are mixed at a 1:1 ratio.

39. A method of treating cancer in a subject comprising administering to the subject the vaccine composition of claim 23
thereby treating cancer in the subject.

40-53. (canceled)

54. The method of claim 39, wherein treating cancer comprises preventing progression of cancer in the subject.

55. The method of claim 39, wherein the cancer comprises TARP expressing cancer cells.

56. The method of claim 39, wherein the cancer is prostate cancer, breast cancer or acute myeloid leukemia (AML).

57. The method of claim 39, further comprising administering to the subject an anti-cancer treatment comprising surgery, radiotherapy, chemotherapy, immunotherapy, targeted therapy, or any combination thereof.

58. (canceled)

59. The method of claim 57, wherein the immunotherapy comprises an immune checkpoint inhibitor therapy.

60. The method of claim 59, wherein the checkpoint inhibitor therapy comprises a programmed cell death 1 protein (PD-1) inhibitor, a PD-1 ligand 1 (PD-L1) inhibitor, and/or a cytotoxic T-lymphocyte-associated protein 4 (CTLA-4) inhibitor.

61. The method of claim 57, wherein the targeted therapy comprises a histone deacetylase (HDAC) inhibitor.

62. (canceled)

63. A method of inducing a TARP-specific polyfunctional cytolytic T cell response in the subject, comprising administering to the subject the vaccine composition of claim 23
thereby inducing a TARP-specific polyfunctional cytolytic T cell response in the subject.

64-77. (canceled)
